(19) United States
(12) Patent Application Publication
(10) Pub. No.: US 2026/0183241 A1
(43) Pub. Date: Jul. 2, 2026

(54) PHARMACEUTICAL COMPOSITIONS OF ISAVUCONAZOLE, PREPARATION METHODS AND USES THEREOF

(71) Applicant: Hefei Cosource Pharmaceuticals Co., Ltd, Hefei (CN)

(72) Inventors: Jun ZHANG, Hefei (CN); Guodong CHENG, Hefei (CN); Dengjun CHEN, Hefei (CN); Yuting JIN, Hefei (CN); Liuyu SHI, Hefei (CN); Guixia CHEN, Hefei (CN); Jieyong CAO, Hefei (CN); Xujun FANG, Hefei (CN); Chengjing LU, Hefei (CN); Nan LI, Hefei (CN)

(21) Appl. No.: 19/437,166

(22) Filed: Dec. 30, 2025

(30) Foreign Application Priority Data
Dec. 31, 2024 (CN) 202411997856.0

(51) Int. Cl. A61K 9/16; A61K 9/00; A61K 31/427

(52) U.S. Cl. CPC A61K 9/1652; A61K 9/0053; A61K 9/1617; A61K 9/1623; A61K 9/1635; A61K 9/1641; A61K 9/1664; A61K 31/427

(57) ABSTRACT

The present disclosure provides a pharmaceutical composition comprising isavuconazole, a first nonionic surfactant, a stabilizer, a degradation inhibitor, and a diluent, wherein the first nonionic surfactant comprises copovidone and poloxamer, a method for preparing the pharmaceutical composition, and a method for treating, ameliorating or preventing related diseases by administering the pharmaceutical composition.

### CROSS-REFERENCE TO RELATED APPLICATIONS

The present application claims the benefit and priority to the Chinese patent application No. 202411997856.0 filed before the China national intellectual property administration on Dec. 31, 2024, the content of which is incorporated herein by reference in its entirety.

### TECHNICAL FIELD

The present disclosure belongs to the field of pharmaceutical formulations, and specifically relates to a pharmaceutical composition and a preparation method and use thereof.

### BACKGROUND

Isavuconazole is difficult to develop into a drug due to its low solubility. The active pharmaceutical ingredient (API) currently developed for the market is a prodrug of isavuconazole, isavuconazonium sulfate, and the marked formulation is isavuconazonium sulfate capsule. In the FDA-approved isavuconazonium sulfate capsules (Cresemba®), the packaging material (aluminum-plastic blister pack) for said formulation incorporates two interconnected compartments in order to reduce the degradation of this compound: one containing the capsule and the other holding a desiccant, thereby meeting long-term storage requirements.

Therefore, the isavuconazonium sulfate capsule formulation needs to be improved.

### SUMMARY

A first aspect of the present disclosure provides a pharmaceutical composition comprising isavuconazole, a first nonionic surfactant, a stabilizer, a degradation inhibitor, and a diluent, wherein the first nonionic surfactant comprises copovidone and poloxamer.

A second aspect of the present disclosure provides a method for preparing the pharmaceutical composition described in the first aspect, comprising mixing isavuconazole, a first nonionic surfactant, a diluent, a stabilizer, a degradation inhibitor, and a solvent, wherein the first nonionic surfactant comprises copovidone and poloxamer.

A third aspect of the present disclosure provides use of the pharmaceutical composition described in the first aspect of the present disclosure in the preparation of a medicament for treating, ameliorating or preventing related diseases, wherein the related diseases include fungal urinary tract infection, candidal vaginitis, aspergillosis or mucormycosis.

A fourth aspect of the present disclosure provides a method for treating, ameliorating or preventing related diseases, comprising administering to a subject in need thereof the pharmaceutical composition described in the first aspect of the present disclosure, wherein the related diseases comprise fungal urinary tract infection, candidal vaginitis, aspergillosis, or mucormycosis.

Additional aspects and advantages of the present disclosure will be set forth in part in the following description, or will become apparent in part from the following description, or will be understood through practice of the present disclosure.

### BRIEF DESCRIPTION OF THE DRAWINGS

In order to illustrate more clearly specific embodiments of the present disclosure or the technical solutions in the prior art, the drawings thar are required for describing the specific embodiments or the prior art will be briefly described below. It is apparent that the drawings described below belong to some embodiments of the present disclosure. It is also apparent for a person skilled in the art that other drawings may be derived from these drawings without involving any creative effort.

FIG. 1 shows the mean concentration of the drug in plasma-time curves for isavuconazole active pharmaceutical ingredient (Group 1), isavuconazonium sulfate capsule (Group 2), the pharmaceutical composition of Example 1 (Group 3), and the pharmaceutical composition of Example 2 (Group 4).

FIG. 2 shows the mean concentration of the drug in the kidney-time curves for isavuconazole active pharmaceutical ingredient (Group 1), isavuconazonium sulfate capsule (Group 2), the pharmaceutical composition of Example 1 (Group 3), and the pharmaceutical composition of Example 2 (Group 4);

FIG. 3 shows the mean concentration of the drug in the bladder-time curves for isavuconazole active pharmaceutical ingredient (Group 1), isavuconazonium sulfate capsule (Group 2), the pharmaceutical composition of Example 1 (Group 3), and the pharmaceutical composition of Example 2 (Group 4);

FIG. 4 shows the mean concentration of the drug in urine-time curves for isavuconazole active pharmaceutical ingredient (Group 1), isavuconazonium sulfate capsule (Group 2), the pharmaceutical composition of Example 1 (Group 3), and the pharmaceutical composition of Example 2 (Group 4).

### DETAILED DESCRIPTION OF THE INVENTION

To clarify the objectives, technical solutions, and advantages of the present disclosure, the technical solutions of the present disclosure will be described in detail below. Obviously, the described embodiments constitute only a portion of the embodiments of the present disclosure, rather than the entirety of the embodiments. All other embodiments that are arrived at by a person skilled in the art based on the embodiments of the present disclosure without requiring creative effort fall within the protection scope of the present disclosure.

The “range”, as disclosed in the present disclosure, is defined by means of a lower limit and an upper limit. A given range is defined by selecting a lower limit and an upper limit, and the selected lower and upper limits define the boundaries of a particular range. The range defined in this manner may be inclusive of endpoints, and may be combined arbitrarily. That is, any lower limit can be combined with any upper limit to form a range. For example, if the ranges of 60-120 and 80-110 are specified for a specific parameter, then it should be understood that the ranges of 60-110 and 80-120 are also encompassed. Furthermore, for a range where the minimum values are listed as 1 and 2 and the maximum values are listed as 3, 4 and 5, the following ranges can all be anticipated: 1-3, 1-4, 1-5, 2-3, 2-4 and 2-5. In the present disclosure, unless specified otherwise, the numerical range “a-b” indicates an abbreviated representation for combinations of any real numbers between a and b, where both a and b are real numbers, and the range defined in this way may be inclusive of the endpoints a and b. For example, the numerical range “0-5” indicates that all real numbers between 0 and 5 are exhaustively listed herein, and “0-5” is merely an abbreviated representation of these numerical combinations. Additionally, when a parameter is expressed as an integer ≥2, it is equivalent to disclosing that the parameter encompasses an integer, such as 2, 3, 4, 5, 6, 7, 8, 9, 10, 11, 12, and so on.

It should be noted that the terms “first” and “second” are used solely for descriptive purposes, and should not be construed as indicating or implying relative importance or implying the number of technical features referred to. Therefore, the features defined by the terms “first” and “second” may explicitly or implicitly include one or more instances of the features. Furthermore, in the description of the present disclosure, the term “multiple” refers to at least two, such as two, three, and so on, unless otherwise explicitly and specifically defined.

All embodiments and optional embodiments of the present disclosure may be combined with one another to form a new technical solution, unless otherwise specified.

All technical features and optional technical features of the present disclosure may be combined with one another to form a new technical solution, unless otherwise specified.

In a first aspect of the present disclosure, the present disclosure provides a pharmaceutical composition comprising isavuconazole, a first nonionic surfactant, a stabilizer, a degradation inhibitor, and a diluent, wherein the first nonionic surfactant comprises copovidone and poloxamer.

By mixing isavuconazole with a first nonionic surfactant comprising copovidone and poloxamer, as well as a diluent, a degradation inhibitor and a stabilizer, not only can the solubility of isavuconazole be significantly improved, but also the long-term storage stability of isavuconazole can be improved.

In some embodiments of the present disclosure, in the above pharmaceutical composition, a mass ratio of the isavuconazole, the copovidone, the poloxamer, the diluent, the stabilizer, and the degradation inhibitor is 1:2.5-4.5:0.45-1:3-5:3.5-5.5:0.25-2, such as 1:2.5:0.45-1:3-5:3.5-5.5:0.25-2, 1:3:0.45-1:3-5:3.5-5.5:0.25-2, 1:3.5:0.45-1:3-5:3.5-5.5:0.25-2, 1:4:0.45-1:3-5:3.5-5.5:0.25-2, 1:4.5:0.45-1:3-5:3.5-5.5:0.25-2, 1:2.5-4.5:0.45:3-5:3.5-5.5:0.25-2, 1:2.5-4.5:0.5:3-5:0.25-2, 1:2.5-4.5:0.7:3-5:3.5-5.5:0.25-2, 1:2.5-4.5:1:3-5:3.5-5.5:0.25-2, 1:2.5-4.5:0.45-1:3:3.5-5.5:0.25-2, 1:2.5-4.5:0.45-1:4:3.5-5.5:0.25-2, 1:2.5-4.5:0.45-1:5:3.5-5.5:0.25-2, 1:2.5-4.5:0.45-1:3-5:3.5:0.25-2, 1:2.5-4.5:0.45-1:3-5:4:0.25-2, 1:2.5-4.5:0.45-1:3-5:4.5:0.25-2, 1:2.5-4.5:0.45-1:3-5:5:0.25-2, 1:2.5-4.5:0.45-1:3-5:5.5:0.25-2, 1:2.5-4.5:0.45-1:3-5:3.5-5.5:0.25, 1:2.5-4.5:0.45-1:3-5:3.5-5.5:0.5, 1:2.5-4.5:0.45-1:3-5:3.5-5.5:0.7, 1:2.5-4.5:0.45-1:3-5:3.5-5.5:1, 1:2.5-4.5:0.45-1:3-5:3.5-5.5:1.25, 1:2.5-4.5:0.45-1:3-5:3.5-5.5:1.5, 1:2.5-4.5:0.45-1:3-5:3.5-5.5:1.7, 1:2.5-4.5:0.45-1:3-5:3.5-5.5:2, and so on. By mixing isavuconazole, copovidone, poloxamer, stabilizer, degradation inhibitor, and diluent in the aforementioned ratios, not only can the solubility of isavuconazole be further significantly improved, but also the long-term storage stability of isavuconazole can be further markedly improved.

As an example, the diluent comprises one or more of crospovidone, microcrystalline cellulose, silicon dioxide, pregelatinized starch, corn starch, lactose, xylitol, and mannitol.

As an example, the stabilizer comprises one or more of Hypromellose E5, Hypromellose E50, Hypromellose K100LV, Hypromellose E4M, Hypromellose K4M, hydroxypropyl cellulose LF, hydroxypropyl cellulose LXF, hydroxypropyl cellulose JF, hydroxypropyl cellulose JXF, hydroxypropyl cellulose HXF, hydroxypropyl cellulose GXF, and hydroxypropyl cellulose GF. Furthermore, the stabilizer comprises one or more of Hypromellose E5 and Hypromellose E50.

As an example, the degradation inhibitor comprises one or more of anhydrous citric acid, anhydrous citric acid-sodium hydroxide, monosodium citrate, tartaric acid-sodium hydroxide, sodium bitartrate, succinic acid-sodium hydroxide, sodium bisuccinate, malic acid-sodium hydroxide, monosodium malate, benzoic acid, and citric acid-trisodium citrate.

In some embodiments of the present disclosure, the pharmaceutical composition may further comprise a second nonionic surfactant. The second nonionic surfactant comprises one or more of Polyoxyl(40)Hydrogenated Castor Oil (RH40), TPGS (vitamin E polyethylene glycol succinate), and HS15 (polyethylene glycol (15)-hydroxy stearate). The second nonionic surfactant can further increase the solubility of isavuconazole, and can further improve the long-term storage stability of isavuconazole.

In some embodiments of the present disclosure, a mass ratio of the isavuconazole to the second nonionic surfactant in the above-mentioned pharmaceutical composition is 1:0.1-2, such as 1:0.1, 1:0.3, 1:0.5, 1:0.7, 1:1, 1:1.3, 1:1.5, 1:1.7, 1:2, and so on, or may be within any range defined by any two of the aforementioned values. By adding said amount of the second nonionic surfactant to the above-mentioned pharmaceutical composition, not only can the solubility of isavuconazole be further improved, but also the long-term storage stability of isavuconazole can be further improved.

In some embodiments of the present disclosure, the above-mentioned pharmaceutical composition may further comprise an ionic surfactant. A mass ratio of the isavuconazole to the ionic surfactant is 1:0.5-1, such as 1:0.5, 1:0.7, 1:1, and so on. By adding the ionic surfactant to the above-mentioned pharmaceutical composition, the solubility and long-term storage stability of isavuconazole can be further improved.

As an example, the ionic surfactant comprises one or more of sodium glycocholate, lecithin, and sodium lauryl sulfate.

In some embodiments of the present disclosure, the above-mentioned pharmaceutical composition may further comprise one or more of a flavoring agent, a flavor, and an oily solvent.

In some embodiments of the present disclosure, when the pharmaceutical composition comprises a flavoring agent, a mass ratio of the isavuconazole to the flavoring agent is 1:0.1-2, such as 1:0.1, 1:0.3, 1:0.5, 1:0.8, 1:1, 1:1.3, 1:1.5, 1:1.8, 1:2, and so on, or may be within any range defined by any two of the aforementioned values.

In some embodiments of the present disclosure, when the pharmaceutical composition comprises a flavor, the flavor is present at a content of 0.01% to 2%, such as 0.01%, 0.03%, 0.05%, 0.07%, 0.1%, 0.3%, 0.5%, 0.7%, 1%, 1.3%, 1.5%, 1.7%, 2%, and so on, based on the total mass of the pharmaceutical composition, or may be within any range defined by any two of the aforementioned values.

In some embodiments of the present disclosure, when the pharmaceutical composition comprises an oily solvent, the oily solvent is present at a content of 2% to 6%, such as 2%, 2.5%, 3%, 3.5%, 4%, 4.5%, 5%, 5.5%, 6%, and so on, based on the total mass of the pharmaceutical composition, or may be within any range defined by any two of the aforementioned values.

As an example, the flavoring agent comprises one or more of sucralose, xylitol, mannitol, and sorbitol. As an example, the flavor comprises one or more of strawberry flavor, cherry flavor, sweet orange flavor, apple flavor, banana flavor, chocolate flavor, and mango flavor. As an example, the oily solvent comprises one or more of medium-chain triglycerides, triethyl citrate, castor oil, triacetin, soybean oil, sesame oil, peanut oil, corn oil, and olive oil.

In some embodiments of the present disclosure, a dosage form of the pharmaceutical composition includes granules, a tablet, a powder, an emulsion, a solution, or a suspension.

It should be noted that when the pharmaceutical composition is formulated as tablets, powders, emulsions, solutions or suspensions, a person skilled in the art can add an excipient suitable for preparing said dosage form as needed.

In a second aspect of the present disclosure, the present disclosure provides a method for preparing the aforementioned pharmaceutical composition, comprising mixing isavuconazole, a first nonionic surfactant, a diluent, a stabilizer, a degradation inhibitor, and a solvent, wherein the first nonionic surfactant comprises copovidone and poloxamer.

In the present disclosure, not only can the solubility of isavuconazole be significantly improved, but also the long-term storage stability of isavuconazole can be improved, through mixing isavuconazole, the first nonionic surfactant comprising copovidone and poloxamer, the stabilizer, the degradation inhibitor and the diluent.

In some embodiments of the present disclosure, when the dosage form of the pharmaceutical composition is granules, wet granulation may be employed. For example, a wet granulation process comprises:
(1) dissolving the prescribed amounts of isavuconazole, copovidone, and poloxamer and optionally a second nonionic surfactant in a solvent, and adding the prescribed amount of a degradation inhibitor to obtain a binder;
(2) mixing a diluent, a stabilizer, optionally a flavoring agent, and optionally an ionic surfactant;
(3) adding a mixture obtained in step (2) to the binder obtained in step (1) to obtain a soft material, or adding the binder obtained in step (1) to a mixture obtained in step (2) to obtain a soft material;
(4) granulating the soft material to obtain wet granules;
(5) drying the wet granules, and then optionally mixing the dried granules with a flavor to obtain the pharmaceutical composition in granule form.

In some embodiments of the present disclosure, when the dosage form of the pharmaceutical composition is granules, a spray granulation process may be employed. For example, a spray granulation process comprises:
(1) dissolving the prescribed amounts of isavuconazole, copovidone, and poloxamer and optionally a second nonionic surfactant in a solvent, and adding the prescribed amount of a degradation inhibitor to obtain a binder;
(2) mixing a diluent, a stabilizer, optionally a flavoring agent, and optionally an ionic surfactant, and then fluidizing to obtain a mixture;
(3) spraying the mixture obtained in step (2) onto the binder obtained in step (1), or spraying the binder obtained in step (1) onto the mixture obtained in step (2);
(4) drying the resulting granules after the spraying in step (3) is completed, and then optionally mixing the dried granules uniformly with a flavor to obtain the pharmaceutical composition in granule form.

In some embodiments of the present disclosure, the pharmaceutical composition of the present disclosure and methods for preparing the same have one or more of the following advantages.

The pharmaceutical composition of the present disclosure exhibits higher solubility and long-term storage stability, thereby solving the problem of low solubility of existing isavuconazole formlations.

The pharmaceutical composition of the present disclosure can be formulated as granules, tablets, powders, emulsions, solutions, or suspensions, thereby overcoming the limitation of being available solely as capsules in the prior art. The pharmaceutical composition in granule form can be administered orally or via a nasogastric tube after direct dissolution in water during clinical use, thereby addressing the clinical needs of patients who are unwilling to take capsules orally or have dysphagia.

The pharmaceutical composition of the present disclosure has higher bioavailability.

The preparation process of the existing prodrug isavuconazonium sulfate is complex, and complex and costly procedures such as column chromatography and vacuum freeze-drying are used in the preparation of the API, resulting in extremely high production costs. In contrast, the preparation process of the pharmaceutical composition of the present disclosure is simple and relatively low-cost.

The prodrug isavuconazonium sulfate is metabolized in vivo under the action of esterases to release acetaldehyde and 1-methyl-1H-pyridino[2,3-D][1,3]oxazine-2(4H)-one (Code Name: BAL-8728). Acetaldehyde is a potent carcinogen, and 1-methyl-1H-pyridino[2,3-D][1,3]oxazine-2(4H)-one will increase an additional metabolic burden on the liver and kidney. In contrast, the pharmaceutical composition of the present disclosure does not release acetaldehyde as a carcinogen or [2,3-D][1,3]oxazine-2(4H)-one during metabolism, thereby avoiding the metabolic burden on the kidney.

The pharmaceutical composition of the present disclosure is a self-assembled nanodispersion solid pharmaceutical composition of isavuconazole with enhanced bioavailability.

It should be noted that the features and advantages described above with respect to the pharmaceutical composition are also applicable to the methods for preparing the pharmaceutical composition, and will not be repeated herein.

In a third aspect of the present disclosure, the present disclosure provides use of the pharmaceutical composition described in the first aspect of the present disclosure in the preparation of a medicament for treating, ameliorating or preventing related diseases, wherein the related diseases include fungal urinary tract infection, candidal vaginitis, aspergillosis or mucormycosis.

It should be noted that the features and advantages described above with respect to the pharmaceutical composition and the methods for preparing the same are also applicable to the use of the pharmaceutical composition in the preparation of a medicament for treating, ameliorating or preventing related diseases, and will not be repeated herein.

### EXAMPLES

The examples described below are illustrative and are intended to interpret the present disclosure only, and should not be construed as a limitation to the scope of the present disclosure. Where specific techniques or conditions are not explicitly described in the examples, they shall be understood to follow techniques or conditions as described in related technical references in the art or as per the product specifications. Reagents or the instruments for which manufacturers are not specified are understood to be commercially available conventional products.

### Effects of Copovidone and Poloxamer on Pharmaceutical Composition

### Example 1

A process for preparing a pharmaceutical composition in granular form comprises:
(1) weighing isavuconazole, copovidone, and poloxamer according to Table 1, and dissolving the resulting mixture in ethanol with stirring, and setting aside for later use;
(2) adding sodium hydroxide to purified water and stirring until dissolved, and then adding anhydrous citric acid thereto and stirring in a hot water bath until dissolved, further stirring at room temperature (25° C.) until crystallization occurred, yielding monosodium citrate suspension, and then adding the suspension to the solution obtained in step (1), stirring and dispersing to obtain a binder;
(3) weighing xylitol, Hypromellose, crospovidone and sucralose according to Table 1, blending evently, and adding the binder to form a soft material, and then granulating through a 24-mesh sieve to yield wet granules; and
(4) drying the wet granules in a hot-air circulation oven until moisture content (loss on drying)≤3 wt %, and then sieving through a 24-mesh sieve to obtain the pharmaceutical composition in granular form.

### Comparative Examples 1-2

The preparation processes in Comparative Examples 1-2 were the same as that in Example 1, with the differences shown in Table 1.

### Comparative Example 3

A process for preparing a pharmaceutical composition in granular form comprises:
(1) weighing isavuconazole and copovidone according to Table 1, and dissolving the resulting mixture in ethanol with stirring, and setting aside for later use;
(2) crushing monosodium citrate and sieving through a 200-mesh sieve, then adding it to the solution obtained in step (1), stirring and dispersing to obtain a binder;
(3) weighing xylitol, Hypromellose, crospovidone and sucralose according to Table 1, blending evently, and adding the binder to form a soft material, and then granulating through a 24-mesh sieve to yield wet granules; and
(4) drying the wet granules in a hot-air circulation oven until moisture content (loss on drying)≤3 wt %, and then sieving through a 24-mesh sieve to obtain the pharmaceutical composition in granular form.

### Comparative Example 4

The preparation process in Comparative Example 4 was the same as that in Comparative Example 3, with the exception that copovidone was replaced with Polyoxyl(40)Hydrogenated Castor Oil (RH40), and the amount of each component added was as shown in Table 1.

### Comparative Example 5

The preparation process in Comparative Example 5 was the same as that in Comparative Example 3, with the exception that TPGS was also added in step (1), and the amount of each component added was as shown in Table 1.

### Comparative Example 6

The preparation process in Comparative Example 6 was the same as that in Comparative Example 4, with the exception that Polyoxyl(40)Hydrogenated Castor Oil (RH40) was replaced with TPGS, and the amount of each component added was as shown in Table 1.

TABLE 1

| | Performulation (mg/pouch) | | | | | | |
|---|---|---|---|---|---|---|---|
| | | | | Comparative | Comparative | Comparative | Comparative |
| | Example | Comparative | Comparative | Example | Example | Example | Example |
| Component | 1 | Example 1 | Example 2 | 3 | 4 | 5 | 6 |
| Isavuconazole/mg | 104.10 | 104.10 | 104.10 | 104.10 | 104.10 | 104.10 | 104.10 |
| Copovidone/mg | 300.00 | 300.00 | / | 300.00 | / | 300.00 | / |
| Poloxamer P407/mg | 75.00 | / | 75.00 | / | / | / | / |
| Anhydrous citric acid/mg | 44.86 | 44.86 | 44.86 | / | / | / | / |
| Sodium hydroxide/mg | 9.34 | 9.34 | 9.34 | / | / | / | / |
| Purified water | About | About | About | / | / | / | / |
| | 0.025 ml | 0.025 ml | 0.025 ml | | | | |
| Polyoxyl(40)Hydrogenated | / | / | / | / | 75.00 | / | / |
| Castor Oil (RH40)/mg | | | | | | | |
| TPGS/mg | / | / | / | / | / | 75.00 | 75.00 |
| Monosodium citrate/mg | / | / | / | 50.00 | 50.00 | 50.00 | 50.00 |
| Ethanol | About | About 0.62 ml | About 0.62 ml | About | About | About | About |
| | 0.62 ml | | | 0.6 ml | 0.6 ml | 0.6 ml | 0.6 ml |
| Xylitol (sieved through an | 1141.70 | 1216.70 | 1441.70 | 1220.90 | 1445.90 | 1145.90 | 1445.90 |
| 80-mesh sieve)/mg | | | | | | | |
| Hypromellose E5/mg | 450.00 | 450.00 | 450.00 | 450.00 | 450.00 | 450.00 | 450.00 |
| Crospovidone XL-10/mg | 350.00 | 350.00 | 350.00 | 350.00 | 350.00 | 350.00 | 350.00 |
| Sucralose ((sieved through | 25.00 | 25.00 | 25.00 | 25.00 | 25.00 | 25.00 | 25.00 |
| an 80-mesh sieve)/mg | | | | | | | |
| Total amount of | 2500.00 | 2500.00 | 2500.00 | 2500 | 2500 | 2500 | 2500 |
| pharmaceutical composition/mg | | | | | | | |

### Effects of Copovidone and Poloxamer at Different Mixing Ratios on Pharmaceutical Composition

### Example 2

A process for preparing a pharmaceutical composition in granular form comprises:
(1) weighing isavuconazole, copovidone, and poloxamer according to Table 2, and dissolving the resulting mixture in ethanol with stirring, and setting aside for later use;
(2) adding sodium hydroxide to purified water and stirring until dissolved, and then adding tartaric acid thereto and stirring in a hot water bath until dissolved, further stirring at room temperature (25° C.) until crystallization occurred, yielding sodium bitartrate, and then adding the resulting mixture to the solution obtained in step (1), stirring and dispersing to obtain a binder;
(3) weighing xylitol, Hypromellose, crospovidone and sucralose according to Table 2, blending evently, and adding the binder to form a soft material, and then granulating through a 24-mesh sieve to yield wet granules; and
(4) drying the wet granules in a hot-air circulation oven until moisture content (loss on drying)≤3 wt %, and then sieving through a 24-mesh sieve to obtain the pharmaceutical composition in granular form.

### Example 3

The preparation process in Example 3 was the same as that in Example 2, with the differences shown in Table 2.

TABLE 2

| | Performulation (mg/pouch) | |
|---|---|---|
| Component | Example 2 | Example 3 |
| Isavuconazole/mg | 104.10 | 104.10 |
| Copovidone/mg | 300.00 | 300.00 |
| Poloxamer P407/mg | 50.00 | 100.00 |
| Tartaric acid/mg | 34.89 | 34.89 |
| Sodium hydroxide/mg | 9.30 | 9.30 |
| Purified water | About 0.025 ml | About 0.025 ml |
| Ethanol | About 0.6 ml | About 0.6 ml |
| Xylitol (sieved through | 1176.71 | 1126.71 |
| an 80-mesh sieve)/mg | | |
| Hypromellose E5/mg | 450.00 | 450.00 |
| Crospovidone XL-10/mg | 350.00 | 350.00 |
| Sucralose (sieved through | 25.00 | 25.00 |
| an 80-mesh sieve)/mg | | |
| Total amount of | 2500.00 | 2500.00 |
| pharmaceutical composition/mg | | |

Effects of nonionic surfactants Polyoxyl(40)Hydrogenated Castor Oil (RH40), TPGS and HS15 on Pharmaceutical Composition

### Example 4

A process for preparing an oral pharmaceutical composition comprising isavuconazole in granular form comprises:
(1) weighing isavuconazole, copovidone, poloxamer and Polyoxyl(40)Hydrogenated Castor Oil (RH40) according to Table 3, and dissolving the resulting mixture in ethanol with stirring, and setting aside for later use;
(2) crushing monosodium citrate and sieving through a 200-mesh sieve, then adding it to the solution obtained in step (1), stirring and dispersing to obtain a binder;
(3) weighing xylitol, Hypromellose E5, crospovidone and sucralose according to Table 3, blending evently, and adding the binder to form a soft material, and then granulating through a 24-mesh sieve to yield wet granules; and
(4) drying the wet granules in a hot-air circulation oven until moisture content (loss on drying)≤3 wt %, and then sieving through a 24-mesh sieve to obtain the pharmaceutical composition in granular form.

### Example 5

A process for preparing an oral pharmaceutical composition comprising isavuconazole in granular form comprises:
(1) weighing isavuconazole, copovidone, poloxamer and TPGS according to Table 3, and dissolving the resulting mixture in ethanol with stirring, and setting aside for later use;
(2) adding sodium hydroxide to purified water and stirring until dissolved, and then adding tartaric acid thereto and stirring in a hot water bath until dissolved, further stirring at room temperature (25° C.) until crystallization occurred, yielding sodium bitartrate, and then adding the resulting mixture to the solution obtained in step (1), stirring and dispersing to obtain a binder;
(3) weighing xylitol, Hypromellose E50, crospovidone and sucralose according to Table 3, blending evently, and adding the binder to form a soft material, drying the soft material to a semi-dry state, and then granulating through a 24-mesh sieve to yield granules; and
(4) drying the granules obtained in step (3) in a hot-air circulation oven until moisture content (loss on drying)≤3 wt %, and then sieving through a 24-mesh sieve to obtain the pharmaceutical composition in granular form.

### Example 6

The preparation process in Example 6 was the same as that in Example 5, with the exception that tartaric acid was replaced with DL-malic acid, Hypromellose E50 was replaced with hydroxypropyl cellulose LXF, and the amount of each component was as shown in Table 3.

### Example 7

The preparation process in Example 7 was the same as that in Example 4, with the exception that Polyoxyl(40)Hydrogenated Castor Oil (RH40) in step (1) was replaced with polyethylene glycol (15)-hydroxy stearate (HS15, SolutolHS15), and the amount of each component was as shown in Table 3.

TABLE 3

| | Performulation (mg/pouch) | | | |
|---|---|---|---|---|
| | Exam- | Exam- | Exam- | Exam- |
| Component | ple 4 | ple 5 | ple 6 | ple 7 |
| Isavuconazole/mg | 104.10 | 104.10 | 104.10 | 104.10 |
| Copovidone/mg | 350.00 | 250.00 | 400.00 | 450.00 |
| Poloxamer P407/mg | 90.00 | 100.00 | 50.00 | 65.00 |
| Polyoxyl(40)Hydrogenated | 100.00 | / | / | / |
| Castor Oil (RH40)/mg | | | | |
| TPGS/mg | / | 100.00 | 100.00 | / |
| Polyethylene glycol (15)- | / | / | / | 100.00 |
| hydroxy stearate (HS15)/mg | | | | |
| Monosodium citrate/mg | 50.00 | / | / | 50.00 |
| Tartaric acid/mg | / | 34.89 | / | / |
| DL-malic acid/mg | / | / | 30.93 | / |
| Sodium hydroxide/mg | / | 9.30 | 9.22 | / |
| Purified water | / | About | About | / |
| | | 0.025 ml | 0.025 ml | |
| Ethanol | About | About | About | About |
| | 0.6 ml | 0.6 ml | 0.6 ml | 0.6 ml |
| Xylitol (sieved through an | 980.90 | 1076.71 | 980.75 | 905.90 |
| 80-mesh sieve)/mg | | | | |
| Hypromellose E5/mg | 450.00 | / | / | 450.00 |
| Hypromellose E50/mg | / | 450.00 | / | / |
| Hydroxypropyl | / | / | 450.00 | / |
| celluloseLXF/mg | | | | |
| Crospovidone XL-10/mg | 350.00 | 350.00 | 350.00 | 350.00 |
| Sucralose (sieved through an | 25.00 | 25.00 | 25.00 | 25.00 |
| 80-mesh sieve)/mg | | | | |
| Total amount of | 2500.00 | 2500.00 | 2500.00 | 2500.00 |
| pharmaceutical | | | | |
| composition/mg | | | | |

Effects of degradation inhibitor on Pharmaceutical Composition

### Comparative Example 7

A process for preparing a pharmaceutical composition in granular form comprises:
(1) weighing isavuconazole, copovidone and poloxamer according to Table 4, and dissolving the resulting mixture in ethanol with stirring, and setting aside for later use;
(2) weighing xylitol, Hypromellose E5, crospovidone and sucralose according to Table 4, blending evently, and adding the mixed solution obtained in step (1) to the resulting mixture, and then granulating through a 24-mesh sieve to yield wet granules; and
(3) drying the wet granules in a hot-air circulation oven until moisture content (loss on drying)≤3 wt %, and then sieving through a 24-mesh sieve to obtain the pharmaceutical composition in granular form.

### Comparative Example 8

The preparation process in Comparative Example 8 was the same as that in Comparative Example 7, with the exception that succinic acid was also added in step (1), and the amount of each component was as shown in Table 4.

### Comparative Example 9

The preparation process in Comparative Example 9 was the same as that in Comparative Example 7, with the exception that DL-malic acid was also added in step (1), and the amount of each component was as shown in Table 4.

### Example 8

A process for preparing an oral pharmaceutical composition comprising isavuconazole in granular form comprises:
(1) weighing isavuconazole, copovidone, and poloxamer according to Table 4, and dissolving the resulting mixture in ethanol with stirring, and setting aside for later use;
(2) adding sodium hydroxide to purified water and stirring until dissolved, and then adding succinic acid thereto and stirring in a hot water bath until dissolved, further stirring at room temperature (25° C.) until crystallization occurred, yielding sodium bisuccinate, and then adding the resulting mixture to the solution obtained in step (1), stirring and dispersing to obtain a binder;
(3) weighing xylitol, Hypromellose, crospovidone and sucralose according to Table 4, blending evently, and adding the binder to form a soft material, and then granulating through a 24-mesh sieve to yield wet granules; and
(4) drying the wet granules in a hot-air circulation oven until moisture content (loss on drying)≤3 wt %, and then sieving through a 24-mesh sieve to obtain the pharmaceutical composition in granular form.

### Example 9

A process for preparing a pharmaceutical composition in granular form comprises:
(1) weighing isavuconazole, copovidone, poloxamer and benzoic acid according to Table 4, and dissolving the resulting mixture in ethanol with stirring, and setting aside for later use;
(2) weighing xylitol, Hypromellose, crospovidone and sucralose according to Table 4, blending evently, and adding the mixed solution obtained in step (1) to the resulting mixture, and then granulating through a 24-mesh sieve to yield wet granules; and;
(3) drying the wet granules in a hot-air circulation oven until moisture content (loss on drying)≤3 wt %, and then sieving through a 24-mesh sieve to obtain the pharmaceutical composition in granular form.

### Example 10

A process for preparing an oral pharmaceutical composition comprising isavuconazole in granular form comprises:
(1) weighing isavuconazole, copovidone, and poloxamer according to Table 4, and dissolving the resulting mixture in ethanol with stirring, and setting aside for later use;
(2) adding anhydrous citric acid to the solution obtained in step (1), stirring and dispersing to obtain a binder;
(3) weighing xylitol, Hypromellose, crospovidone and sucralose according to Table 4, blending evently, and adding the binder to form a soft material, and then granulating through a 24-mesh sieve to yield wet granules; and
(4) drying the wet granules in a hot-air circulation oven until moisture content (loss on drying)≤3 wt %, and then sieving through a 24-mesh sieve to obtain the pharmaceutical composition in granular form.

### Example 11

A process for preparing an oral pharmaceutical composition comprising isavuconazole in granular form comprises:
(1) weighing isavuconazole, copovidone, and poloxamer according to Table 4, and dissolving the resulting mixture in ethanol with stirring, and setting aside for later use;
(2) adding sodium citrate to purified water and stirring until dissolved, and then adding anhydrous citric acid thereto and stirring in a hot water bath until dissolved, further stirring at room temperature (25° C.) until crystallization occurred, yielding citric acid-trisodium citrate, and then adding the resulting mixture to the solution obtained in step (1), stirring and dispersing to obtain a binder;
(3) weighing xylitol, Hypromellose, crospovidone and sucralose according to Table 4, blending evently, and adding the binder to form a soft material, and then granulating through a 24-mesh sieve to yield wet granules; and
(4) drying the wet granules in a hot-air circulation oven until moisture content (loss on drying)≤3 wt %, and then sieving through a 24-mesh sieve to obtain the pharmaceutical composition in granular form.

TABLE 4

| | Performulation (mg/pouch) | | | | | | |
|---|---|---|---|---|---|---|---|
| | | | | | Comparative | Comparative | |
| | Comparative | Example | Example | Example | Example | Example | Example |
| Component | example 7 | 8 | 9 | 10 | 8 | 9 | 11 |
| Isavuconazole/mg | 104.10 | 104.10 | 104.10 | 104.10 | 104.10 | 104.10 | 104.10 |
| Copovidone/mg | 300.00 | 300.00 | 300.00 | 300.00 | 300.00 | 300.00 | 300.00 |
| Poloxamer P407/mg | 75.00 | 75.00 | 75.00 | 75.00 | 75.00 | 75.00 | 75.00 |
| DL-malic acid/mg | / | / | / | / | / | 30.93 | / |
| Sodium hydroxide/mg | / | 9.14 | / | / | / | / | / |
| Benzoic acid/mg | / | / | 28.00 | / | / | / | / |
| Anhydrous citric acid/mg | / | / | / | 44.86 | / | / | 44.86 |
| Succinic acid/mg | / | 26.98 | / | / | 26.98 | / | / |
| Sodium citrate/mg | / | / | / | / | / | / | 32.04 |
| Purified water | / | About | / | / | / | / | About |
| | | 0.025 ml | | | | | 0.025 ml |
| Ethanol | About 0.6 ml | About | About | About | About | About | About |
| | | 0.6 ml | 0.6 ml | 0.6 ml | 0.6 ml | 0.6 ml | 0.6 ml |
| Xylitol (sieved through an 80- | 1120.90 | 1084.78 | 1092.90 | 1076.04 | 1093.92 | 1089.97 | 1044.00 |
| mesh sieve)/mg | | | | | | | |
| Hypromellose E5/mg | 450.00 | 450.00 | 450.00 | 450.00 | 450.00 | 450.00 | 450.00 |
| Crospovidone XL-10/mg | 350.00 | 350.00 | 350.00 | 350.00 | 350.00 | 350.00 | 350.00 |
| Sucralose (sieved through an 80- | 25.00 | 25.00 | 25.00 | 25.00 | 25.00 | 25.00 | 25.00 |
| mesh sieve)/mg | | | | | | | |
| Sodium dodecyl sulfate/mg | 75.00 | 75.00 | 75.00 | 75.00 | 75.00 | 75.00 | 75.00 |
| Total amount of pharmaceutical | 2500.00 | 2500.00 | 2500.00 | 2500.00 | 2500.00 | 2500.00 | 2500.00 |
| composition/mg | | | | | | | |

### Effects of Composition of Stabilizer on Pharmaceutical Composition

### Example 12

A process for preparing an oral pharmaceutical composition comprising isavuconazole in granular form comprises:
(1) weighing isavuconazole, copovidone, poloxamer and TPGS according to Table 5, and dissolving the resulting mixture in ethanol with stirring, and setting aside for later use;
(2) adding sodium hydroxide to purified water and stirring until dissolved, and then adding DL-malic acid thereto and stirring in a hot water bath until dissolved, further stirring at room temperature (25° C.) until crystallization occurred, yielding monosodium DL-malate, and then adding the resulting mixture to the solution obtained in step (1), stirring and dispersing to obtain a binder;
(3) weighing xylitol, hydroxypropyl cellulose JXF, crospovidone and sucralose according to Table 5, blending evently, and adding the binder to form a soft material, and then granulating through a 24-mesh sieve to yield wet granules; and
(4) drying the wet granules under vacuum until moisture content (loss on drying)≤3 wt %, and then sieving through a 24-mesh sieve to obtain the pharmaceutical composition in granular form.

### Example 13

The preparation process in Example 13 was the same as that in Example 12, with the exception that hydroxypropyl cellulose JXF was replaced with hydroxypropyl cellulose HXF, and the amount of each component was as shown in Table 5.

### Comparative Example 10

The preparation process in Comparative Example 10 was the same as that in Example 12, with the exception that hydroxypropyl cellulose JXF was not added, and the amount of each component was as shown in Table 5.

### Example 14

The preparation process in Example 14 was the same as that in Example 12, with the exception that hydroxypropyl cellulose JXF was replaced with hydroxypropyl cellulose GXF, and the amount of each component was as shown in Table 5.

### Example 15

A process for preparing a pharmaceutical composition in granular form comprises:
(1) weighing isavuconazole, copovidone and poloxamer according to Table 5, and dissolving the resulting mixture in ethanol with stirring, and setting aside for later use;
(2) adding sodium hydroxide to purified water and stirring until dissolved, and then adding tartaric acid thereto and stirring in a hot water bath until dissolved, further stirring at room temperature (25° C.) until crystallization occurred, yielding sodium bitartrate, and then adding the mixture to the solution obtained in step (1), stirring and dispersing to obtain a binder;
(3) weighing xylitol, Hypromellose K100LV, crospovidone and sucralose according to Table 5, blending evently, and adding the binder to form a soft material, and then granulating through a 24-mesh sieve to yield wet granules; and
(4) drying the wet granules in a hot-air circulation oven until moisture content (loss on drying)≤3 wt %, and then sieving through a 24-mesh sieve to obtain the pharmaceutical composition in granular form.

### Example 16

The preparation process in Example 16 was the same as that in Example 15, with the exception that Hypromellose K100LV was replaced with Hypromellose E4M, and the amount of each component was as shown in Table 5.

### Example 17

The preparation process in Example 17 was the same as that in Example 15, with the exception that Hypromellose K100LV was replaced with Hypromellose K4M, and the amount of each component was as shown in Table 5.

TABLE 5

| | Performulation (mg/pouch) | | | | | | |
|---|---|---|---|---|---|---|---|
| | | Comparative | | | | | |
| | Example | Example | Example | Example | Example | Example | Example |
| Component | 13 | 10 | 12 | 14 | 15 | 16 | 17 |
| Isavuconazole/mg | 104.10 | 104.10 | 104.10 | 104.10 | 104.10 | 104.10 | 104.10 |
| Copovidone/mg | 400.00 | 400.00 | 400.00 | 400.00 | 300.00 | 300.00 | 300.00 |
| Poloxamer P407/mg | 50.00 | 50.00 | 50.00 | 50.00 | 75.00 | 75.00 | 75.00 |
| TPGS/mg | 100.00 | 100.00 | 100.00 | 100.00 | / | / | / |
| Tartaric acid/mg | / | / | / | / | 34.89 | 34.89 | 34.89 |
| DL-malic acid/mg | 30.93 | 30.93 | 30.93 | 30.93 | / | / | / |
| Sodium hydroxide/mg | 9.22 | 9.22 | 9.22 | 9.22 | 9.30 | 9.30 | 9.30 |
| Purified water | About | About | About | About | About | About | About |
| | 0.025 ml | 0.025 ml | 0.025 ml | 0.025 ml | 0.025 ml | 0.025 ml | 0.025 ml |
| Ethanol | About | About | About | About | About | About | About |
| | 0.6 ml | 0.6 ml | 0.6 ml | 0.6 ml | 0.6 ml | 0.6 ml | 0.6 ml |
| Xylitol (sieved through an | 980.75 | 1430.75 | 980.75 | 980.75 | 1151.71 | 1151.71 | 1151.71 |
| 80-mesh sieve)/mg | | | | | | | |
| Hydroxypropyl cellulose | / | / | 450.00 | / | / | / | / |
| JXF/mg | | | | | | | |
| Hydroxypropyl cellulose | / | / | / | 450.00 | / | / | / |
| GXF/mg | | | | | | | |
| Hydroxypropyl cellulose | 450.00 | / | / | / | / | / | / |
| HXF/mg | | | | | | | |
| Hypromellose K100LV/mg | / | / | / | / | 450.00 | / | / |
| Hypromellose E4M/mg | / | / | / | / | / | 450.00 | / |
| Hypromellose K4M/mg | / | / | / | / | / | / | 450.00 |
| Crospovidone XL-10/mg | 350.00 | 350.00 | 350.00 | 350.00 | 350.00 | 350.00 | 350.00 |
| Sucralose (sieved through | 25.00 | 25.00 | 25.00 | 25.00 | 25.00 | 25.00 | 25.00 |
| an 80-mesh sieve)/mg | | | | | | | |
| Total amount of | 2500.00 | 2500.00 | 2500.00 | 2500.00 | 2500.00 | 2500.00 | 2500.00 |
| pharmaceutical | | | | | | | |
| composition/mg | | | | | | | |

### Effects of Ionic Surfactant and Flavor on Pharmaceutical Composition

### Example 18

A process for preparing a pharmaceutical composition in granular form comprises:
(1) weighing isavuconazole, copovidone, poloxamer and Polyoxyl(40)Hydrogenated Castor Oil (RH40) according to Table 6, and dissolving the resulting mixture in ethanol with stirring, and setting aside for later use;
(2) crushing monosodium citrate and sieving through a 200-mesh sieve, then adding it to the solution obtained in step (1), stirring and dispersing to obtain a binder;
(3) weighing xylitol, Hypromellose E5, crospovidone XL-10, sodium glycocholate and sucralose according to Table 6, blending evently, and adding the binder to form a soft material, and then granulating through a 24-mesh sieve to yield wet granules; and
(4) drying the wet granules in a hot-air circulation oven until moisture content (loss on drying)≤3 wt %, and sieving through a 24-mesh sieve, then adding strawberry flavor powder and blending evently to obtain the pharmaceutical composition in granular form.

### Examples 19-20

The preparation processes in Examples 19-20 were the same as that in Example 18, with the differences shown in Table 2.

### Example 21

A process for preparing a pharmaceutical composition in granular form comprises:
(1) weighing isavuconazole, copovidone and poloxamer according to Table 6, and dissolving the resulting mixture in ethanol with stirring, and setting aside for later use;
(2) crushing monosodium citrate and sieving through a 200-mesh sieve, then adding it to the solution obtained in step (1), stirring and dispersing to obtain a binder;
(3) weighing xylitol, Hypromellose E5, lactose, sodium dodecyl sulfate and sucralose according to table 6, and adding the mixture to a fluidized bed, initiating the air supply and adjusting the airflow rate to ensure good fluidization of the materials in the vessel, and then blending for about 10 min;
(4) adjusting the spray gun position and setting the inlet air temperature to about 40° C.; upon reaching the material temperature of about 35° C., initiating spray atomization and activating the peristaltic pump to spray the binder and initiate granulation; after the spraying of the binder was completed, adjusting the inlet air temperature to maintain the material temperature at 35-45° C.; further drying until loss on drying≤3.0%; and then sieving through a 20-mesh sieve to obtain dried granules.
(5) adding apple flavor powder to the dried granules and blending evently to obtain the pharmaceutical composition in granular form.

TABLE 6

Performulation (mg/pouch)

| | Exam- | Exam- | Exam- | Exam- |
|---|---|---|---|---|
| Component | ple 18 | ple 19 | ple 20 | ple 21 |
| Isavuconazole/mg | 104.10 | 104.10 | 104.10 | 104.10 |
| Copovidone/mg | 300.00 | 300.00 | 300.00 | 300.00 |
| Poloxamer | 75.00 | 75.00 | 75.00 | 75.00 |
| P407/mg | | | | |
| Polyoxyl(40)Hydro- | 100.00 | 100.00 | 100.00 | / |
| genated Castor Oil | | | | |
| (RH40)/mg | | | | |
| Lecithin/mg | / | 75.00 | / | / |
| Monosodium | 50.00 | 50.00 | 50.00 | 50.00 |
| citrate/mg | | | | |
| Ethanol | About | About | About | About |
| | 0.6 ml | 0.6 ml | 0.6 ml | 0.6 ml |
| Xylitol (sieved | 958.40 | 958.40 | 958.40 | 1058.40 |
| through an 80-mesh | | | | |
| sieve)/mg | | | | |
| Hypromellose | 450.00 | 450.00 | 450.00 | 450.00 |
| E5/mg | | | | |
| Crospovidone XL- | 350.00 | / | / | / |
| 10/mg | | | | |
| Microcrystalline | / | 350.00 | / | / |
| cellulose 105/mg | | | | |
| Corn starch/mg | / | / | 350.00 | / |
| 200 mesh | / | / | / | 350.00 |
| lactose/mg | | | | |
| Sodium dodecyl | / | / | 75.00 | 75.00 |
| sulfate/mg | | | | |
| Sodium | 75.00 | / | / | / |
| glycocholate/mg | | | | |
| Strawberry flavor | 12.50 | / | / | / |
| powder/mg | | | | |
| Cherry flavor | / | 12.50 | / | / |
| powder/mg | | | | |
| Sweet orange flavor | / | / | 12.50 | / |
| powder/mg | | | | |
| Apple flavor | / | / | / | 12.50 |
| powder/mg | | | | |
| Sucralose (sieved | 25.00 | 25.00 | 25.00 | 25.00 |
| through an 80-mesh | | | | |
| sieve)/mg | | | | |
| Total amount of | 2500.00 | 2500.00 | 2500.00 | 2500.00 |
| pharmaceutical | | | | |
| composition/mg | | | | |

### Effects of Oily Solvent on Pharmaceutical Composition

### Example 22

A process for preparing a pharmaceutical composition in granular form comprises:
(1) weighing isavuconazole, copovidone, poloxamer and medium-chain triglycerides according to Table 7, and dissolving the resulting mixture in ethanol with stirring, and setting aside for later use;
(2) adding sodium hydroxide to purified water and stirring until dissolved, and then adding anhydrous citric acid thereto and stirring in a hot water bath until dissolved, further stirring at room temperature (25° C.) until crystallization occurred, yielding monosodium citrate suspension, and then adding the suspension to the solution obtained in step (1), stirring and dispersing to obtain a binder;
(3) weighing xylitol, Hypromellose, crospovidone and sucralose according to Table 7, blending evently, and adding the binder to form a soft material, and then granulating through a 24-mesh sieve to yield wet granules; and
(4) drying the wet granules in a hot-air circulation oven until moisture content (loss on drying)≤3 wt %, and then sieving through a 24-mesh sieve to obtain the pharmaceutical composition in granular form.

### Example 23

The preparation process in Example 23 was the same as that in Example 22, with the exception that medium-chain triglycerides in step (1) was replaced with triethyl citrate, and the amount of each component was as shown in Table 7.

TABLE 7

| | Performulation (mg/pouch) | |
|---|---|---|
| Component | Example 22 | Example 23 |
| Isavuconazole/mg | 104.10 | 104.10 |
| Copovidone/mg | 300.00 | 300.00 |
| Poloxamer P407/mg | 75.00 | 75.00 |
| Medium-chain triglycerides/mg | 0.10 | 0.00 |
| Triethyl citrate/mg | / | 0.10 |
| Sodium hydroxide/mg | 9.34 | 9.34 |
| Anhydrous citric acid/mg | 44.86 | 44.86 |
| Purified water | About 0.025 ml | About 0.025 ml |
| Ethanol | About 0.6 ml | About 0.6 ml |
| Xylitol (sieved through an 80- | 1066.60 | 1066.60 |
| mesh sieve)/mg | | |
| Hypromellose E5/mg | 450.00 | 450.00 |
| Crospovidone XL-10/mg | 350.00 | 350.00 |
| Sucralose (sieved through an 80- | 25.00 | 25.00 |
| mesh sieve)/mg | | |
| Sodium dodecyl sulfate/mg | 75.00 | 75.00 |
| Total amount of pharmaceutical | 2500.00 | 2500.00 |
| composition/mg | | |

The dissolution, storage stability, particle size distribution and particle size stability, and pharmacokinetics of the pharmaceutical compositions in the form of granules prepared in Examples 1-23 and Comparative Examples 1-10 were evaluated.

### Evaluation Methods:

### 1. Dissolution Test Method:

Dissolution Conditions: 500 ml of phosphate buffer (50 mmol/L) at a pH of 6.8 was used as the dissolution medium, and the paddle was rotated at a speed of 75 rpm. Samples were taken at 15 min, 30 min, 45 min, 60 min, 120 min, 180 min, and 240 min, respectively, and an equal volume of the fresh blank medium was supplemented immediately.

Test Solution: An appropriate amount of the dissolution solution was taken and filtered immediately, and then the filtrate was diluted with an equal volume of acetonitrile, and shaken thoroughly to afford the test solution.

Reference Standard Solution: An appropriate amount of isavuconazole reference standard was taken and weighed accurately, and then dissolved in the solvent [phosphate buffer at a pH of 6.8-acetonitrile (50:50, v/v)], and quantitatively diluted to prepare a solution containing about 0.1 mg/mL of isavuconazole (calculated as C₂₂H₁₇F₂N₅OS).

Measurement Method: 10 μL of the test solution and 10 μL of the reference standard solution were precisely measured, and injected into the liquid chromatograph, respectively. Chromatograms were recorded, and the dissolution was calculated from peak areas using the external standard method.

### Calculation Formula:

$$Dissolution ( \% ) = Concentration \times Medium Volume \div Labeled Amount \times 100 \%$$

$$Cumulative dissolution ( \% ) = {Dissolution}_{n} + {\sum }_{i = 1}^{n - 1} Dissolution \times Sample Volume \div Medium Volume ,$$

wherein n represents the number of samples taken.

Chromatographic Conditions: Octadecylsilane-bonded silica gel was used as a packing material; water-acetonitrile (35:65) was used as a mobile phase; the flow rate was 1.0 ml/min; the detection wavelength was 284 nm; column temperature was 35° C.; and injection volume was 10 μl.

System Suitability Requirement: The number of theoretical plates (N) calculated based on the peak of isavuconazole shall not be less than 4000.

Limits: The capacity shall not fall below 50% of the labeled amount at 30 minutes.

The dissolution test results of the pharmaceutical compositions in granule form as prepared in Examples 1-23 and Comparative Examples 1-10 were shown in Table 8.

TABLE 8

| Formulation | Cumulative Dissolution (%) - Time (min) | | | | | | |
|---|---|---|---|---|---|---|---|
| No. | 15 min | 30 min | 45 min | 60 min | 120 min | 180 min | 240 min |
| Example 1 | 39.15% | 48.53% | 46.52% | 42.01% | 36.58% | 28.22% | 20.90% |
| Comparative | 16.25% | 18.96% | 17.42% | 15.36% | 13.04% | 10.25% | 9.21% |
| Example 1 | | | | | | | |
| Comparative | 20.42% | 24.68% | 25.31% | 20.27% | 15.36% | 11.28% | 10.98% |
| Example 2 | | | | | | | |
| Comparative | 2.57% | 6.35% | 3.12% | Not | 1.24% | Not | Not |
| Example 3 | | | | detected | | detected | detected |
| Comparative | 25.42% | 27.68% | 30.31% | 26.27% | 17.36% | 14.28% | 12.98% |
| Example 4 | | | | | | | |
| Comparative | 36.92% | 47.54% | 47.08% | 40.95% | 37.22% | 29.03% | 19.85% |
| Example 5 | | | | | | | |
| Comparative | 23.57% | 26.23% | 28.76% | 27.92% | 16.55% | 15.43% | 13.80% |
| Example 6 | | | | | | | |
| Example 2 | 35.04% | 45.23% | 42.06% | 40.37% | 33.21% | 22.16% | 14.59% |
| Example 3 | 45.89% | 54.65% | 53.26% | 47.63% | 42.96% | 36.54% | 28.09% |
| Example 4 | 52.64% | 55.72% | 54.87% | 56.29% | 50.32% | 42.13% | 30.27% |
| Example 5 | 47.65% | 53.29% | 54.22% | 52.14% | 47.52% | 36.47% | 28.25% |
| Example 6 | 46.36% | 54.28% | 53.16% | 54.15% | 48.03% | 40.15% | 31.03% |
| Example 7 | 56.59% | 54.52% | 55.38% | 57.04% | 49.65% | 41.88% | 32.63% |
| Example 8 | 74.02% | 75.51% | 76.62% | 76.00% | 76.43% | 71.05% | 65.24% |
| Example 9 | 75.33% | 74.26% | 74.57% | 75.39% | 74.96% | 67.53% | 63.88% |
| Example 10 | 75.97% | 75.11% | 75.75% | 76.27% | 75.93% | 68.99% | 64.57% |
| Example 11 | 75.00% | 74.88% | 76.26% | 75.97% | 76.18% | 69.36% | 64.07% |
| Example 12 | 37.57% | 55.43% | 54.97% | 54.65% | 47.88% | 42.06% | 33.66% |
| Example 13 | 20.62% | 46.58% | 55.26% | 55.93% | 50.62% | 44.85% | 35.72% |
| Example 14 | 30.29% | 50.27% | 56.25% | 57.03% | 48.95% | 43.57% | 34.95% |
| Example 15 | 40.78% | 53.95% | 55.27% | 54.96% | 49.22% | 42.69% | 34.26% |
| Example 16 | 35.89% | 49.04% | 55.27% | 56.01% | 48.31% | 43.54% | 33.75% |
| Example 17 | 26.58% | 44.39% | 54.83% | 57.92% | 50.44% | 44.05% | 36.83% |
| Example 18 | 78.53% | 83.57% | 82.96% | 83.02% | 82.02% | 73.26% | 58.35% |
| Example 19 | 77.65% | 81.29% | 81.98% | 81.66% | 81.59% | 71.47% | 57.56% |
| Example 20 | 80.64% | 82.28% | 82.55% | 81.97% | 82.36% | 72.22% | 58.39% |
| Example 21 | 73.85% | 77.52% | 79.03% | 78.82% | 77.82% | 68.96% | 55.52% |
| Example 22 | 77.18% | 78.62% | 78.96% | 78.52% | 78.86% | 72.21% | 69.97% |
| Example 23 | 76.12% | 77.96% | 77.82% | 78.01% | 77.54% | 70.52% | 68.73% |
| Comparative | 74.51% | 78.42% | 78.06% | 76.59% | 75.13% | 70.05% | 64.55% |
| Example 7 | | | | | | | |
| Comparative | 73.95% | 75.27% | 75.99% | 77.03% | 76.89% | 68.57% | 63.21% |
| Example 8 | | | | | | | |
| Comparative | 75.59% | 75.95% | 75.82% | 76.85% | 75.96% | 67.95% | 62.55% |
| Example 9 | | | | | | | |
| Comparative | 42.99% | 56.84% | 55.46% | 56.27% | 45.98% | 41.88% | 32.06% |
| Example 10 | | | | | | | |

### 2. Particle Size Distribution and Particle Size Stability Test

### Instrument: Nanoparticle Potential Analyzer

### Instrument Parameter Settings

Dispersion medium: Water
Sample cell temperature: 25° C.
Sample cell type: PS sample cell

Preparation of Test Sample: An appropriate amount of granules was taken, and 150 volumes of ultrapure water at ambient temperature was added, and then vortexed rapidly to disperse and dissolve, and filtered through a 0.8 μm water-based microporous membrane to obtain the filtrate. About 1.5 mL of the filtrate was transferred to the sample cell to determine the particle size distribution.

The particle size distribution test results of the pharmaceutical compositions in granule form as prepared in Examples 1-23 and Comparative Examples 1-10 were shown in Table 9.

TABLE 9

| | | | | Number of | | | | |
|---|---|---|---|---|---|---|---|---|
| | | | | Peaks in the | | | | |
| | | | | Light | | | | |
| | | Z-Mean Diameter | | Intensity | D10 | D50 | D90 | |
| Sample | Time | (nm) | PDI | Distribution | nm | nm | nm | Granule dissolution Phenomenon |
| Example 1 | 0 h | 291.52 | 0.136 | 2 | 193.62 | 290.93 | 431.27 | Granules dissolved completely within 30 s of vortexing |
| | 1 h | 296.36 | 0.112 | 2 | 207.54 | 293.42 | 411.04 | |
| | 2 h | 315.73 | 0.219 | 2 | 174.36 | 299.59 | 498.19 | |
| Example 2 | 0 h | 330.64 | 0.227 | 2 | 200.57 | 356.59 | 521.64 | Granules dissolved completely within 30 s of vortexing |
| | 1 h | 337.55 | 0.236 | 2 | 198.36 | 362.47 | 536.92 | |
| | 2 h | 354.83 | 0.254 | 2 | 195.52 | 380.93 | 657.26 | |
| Example 3 | 0 h | 276.66 | 0.147 | 2 | 178.39 | 276.81 | 416.65 | Granules dissolved completely within 30 s of vortexing |
| | 1 h | 281.83 | 0.133 | 2 | 192.08 | 278.06 | 396.88 | |
| | 2 h | 300.65 | 0.207 | 2 | 159.75 | 284.96 | 480.22 | |
| Example 4 | 0 h | 262.47 | 0.158 | 2 | 163.12 | 260.22 | 400.09 | Granules dissolved completely within 30 s of vortexing |
| | 1 h | 267.20 | 0.104 | 2 | 177.28 | 264.20 | 382.26 | |
| | 2 h | 285.05 | 0.228 | 2 | 147.38 | 266.42 | 468.61 | |
| Example 5 | 0 h | 286.65 | 0.188 | 2 | 185.92 | 296.79 | 455.90 | Granules dissolved within about 1 min of vortexing |
| | 1 h | 277.09 | 0.188 | 2 | 165.85 | 291.50 | 479.32 | |
| | 2 h | 287.67 | 0.236 | 2 | 114.93 | 237.69 | 603.26 | |
| Example 6 | 0 h | 272.96 | 0.174 | 2 | 172.12 | 275.47 | 425.04 | Granules dissolved within about 3 min of vortexing |
| | 1 h | 267.42 | 0.182 | 2 | 166.28 | 288.27 | 407.28 | |
| | 2 h | 276.22 | 0.219 | 2 | 156.38 | 279.69 | 449.52 | |
| Example 7 | 0 h | 230.21 | 0.146 | 2 | 135.72 | 245.03 | 346.27 | Granules dissolved completely within 30 s of vortexing |
| | 1 h | 224.06 | 0.139 | 2 | 142.63 | 236.87 | 353.46 | |
| | 2 h | 252.97 | 0.198 | 2 | 127.57 | 256.23 | 422.57 | |
| Example 8 | 0 h | 175.36 | 0.127 | 2 | 79.61 | 170.85 | 314.55 | Granules dissolved completely within 30 s of vortexing |
| | 1 h | 172.55 | 0.113 | 2 | 84.63 | 171.62 | 295.61 | |
| | 2 h | 190.64 | 0.135 | 2 | 60.57 | 184.59 | 367.86 | |
| Example 9 | 0 h | 183.22 | 0.109 | 2 | 85.47 | 175.98 | 300.59 | Granules dissolved completely within 30 s of vortexing |
| | 1 h | 179.54 | 0.121 | 2 | 79.65 | 183.62 | 319.88 | |
| | 2 h | 191.85 | 0.146 | 2 | 62.57 | 200.07 | 375.58 | |
| Example 10 | 0 h | 177.28 | 0.131 | 2 | 80.26 | 173.43 | 325.93 | Granules dissolved completely within 30 s of vortexing |
| | 1 h | 179.69 | 0.109 | 2 | 89.54 | 175.88 | 296.31 | |
| | 2 h | 193.28 | 0.152 | 2 | 60.57 | 170.35 | 399.21 | |
| Example 11 | 0 h | 175.42 | 0.121 | 2 | 78.39 | 181.29 | 301.47 | Granules dissolved completely within 30 s of vortexing |
| | 1 h | 181.67 | 0.132 | 2 | 75.57 | 180.39 | 331.26 | |
| | 2 h | 193.75 | 0.151 | 2 | 63.17 | 198.75 | 393.06 | |
| Example 12 | 0 h | 260.55 | 0.131 | 2 | 132.52 | 257.28 | 387.57 | Granules dissolved within about 5 min of vortexing |
| | 1 h | 257.22 | 0.142 | 2 | 131.74 | 246.53 | 399.26 | |
| | 2 h | 265.36 | 0.149 | 2 | 152.15 | 276.59 | 430.55 | |
| Example 13 | 0 h | 254.06 | 0.142 | 2 | 127.59 | 249.39 | 392.55 | Granules dissolved within about 10 min of vortexing |
| | 1 h | 249.53 | 0.117 | 2 | 143.26 | 255.27 | 400.57 | |
| | 2 h | 260.79 | 0.175 | 2 | 141.57 | 261.95 | 458.21 | |
| Example 14 | 0 h | 255.33 | 0.146 | 2 | 115.27 | 268.75 | 388.59 | Granules dissolved within about 8 min of vortexing |
| | 1 h | 248.95 | 0.138 | 2 | 132.15 | 259.93 | 421.03 | |
| | 2 h | 262.17 | 0.168 | 2 | 148.53 | 280.46 | 468.52 | |
| Example 15 | 0 h | 295.00 | 0.156 | 2 | 195.93 | 322.58 | 486.03 | Granules dissolved within about 5 min of vortexing |
| | 1 h | 307.33 | 0.147 | 2 | 210.26 | 308.95 | 497.96 | |
| | 2 h | 295.49 | 0.165 | 2 | 171.27 | 299.09 | 506.82 | |
| Example 16 | 0 h | 287.55 | 0.164 | 2 | 188.54 | 296.70 | 468.78 | Granules dissolved within about 8 min of vortexing |
| | 1 h | 306.34 | 0.126 | 2 | 212.78 | 320.73 | 468.70 | |
| | 2 h | 299.88 | 0.183 | 2 | 176.35 | 357.57 | 499.76 | |
| Example 17 | 0 h | 299.17 | 0.121 | 2 | 216.27 | 307.52 | 477.41 | Granules dissolved within about 10 min of vortexing |
| | 1 h | 302.82 | 0.133 | 2 | 208.93 | 310.36 | 480.22 | |
| | 2 h | 315.66 | 0.127 | 2 | 220.27 | 330.57 | 497.58 | |
| Example 18 | 0 h | 208.90 | 0.129 | 2 | 96.07 | 204.07 | 341.06 | Granules dissolved completely within 30 s of vortexing |
| | 1 h | 205.85 | 0.126 | 2 | 99.10 | 199.08 | 337.44 | |
| | 2 h | 218.01 | 0.142 | 2 | 96.36 | 220.59 | 388.59 | |
| Example 19 | 0 h | 216.58 | 0.132 | 2 | 90.29 | 206.39 | 349.89 | Granules dissolved completely within 30 s of vortexing |
| | 1 h | 210.77 | 0.121 | 2 | 93.31 | 213.15 | 335.31 | |
| | 2 h | 225.64 | 0.157 | 2 | 88.74 | 206.53 | 389.88 | |
| Example 20 | 0 h | 153.62 | 0.141 | 2 | 56.78 | 148.42 | 288.69 | Granules dissolved completely within 30 s of vortexing |
| | 1 h | 152.26 | 0.152 | 2 | 59.61 | 152.04 | 278.90 | |
| | 2 h | 166.91 | 0.161 | 2 | 42.35 | 160.00 | 355.89 | |
| Example 21 | 0 h | 177.82 | 0.122 | 2 | 81.05 | 176.09 | 310.66 | Granules dissolved completely within 30 s of vortexing |
| | 1 h | 178.36 | 0.121 | 2 | 83.42 | 177.89 | 313.93 | |
| | 2 h | 192.38 | 0.147 | 2 | 62.57 | 188.44 | 385.05 | |
| Example 22 | 0 h | 175.39 | 0.124 | 2 | 79.00 | 176.07 | 308.01 | Granules dissolved completely within 30 s of vortexing |
| | 1 h | 177.11 | 0.123 | 2 | 80.12 | 176.05 | 305.46 | |
| | 2 h | 181.05 | 0.119 | 2 | 75.47 | 179.53 | 311.09 | |
| Example 23 | 0 h | 176.32 | 0.129 | 2 | 79.93 | 172.14 | 296.37 | Granules dissolved completely within 30 s of youtexing |
| | 1 h | 176.12 | 0.111 | 2 | 82.59 | 173.75 | 295.96 | |
| | 2 h | 179.36 | 0.116 | 2 | 78.36 | 177.47 | 314.85 | |
| Comparative | 0 h | 349.72 | 0.264 | 2 | 243.82 | 376.13 | 504.47 | Granules dissolved completely within 30 s of vortexing |
| Example 1 | 1 h | 375.56 | 0.275 | 3 | 257.74 | 395.62 | 892.24 | |
| | 2 h | 446.93 | 0.301 | 3 | 224.56 | 432.79 | 2036.7 | |
| Comparative | 0 h | 315.62 | 0.145 | 2 | 213.72 | 328.53 | 462.87 | Granules dissolved completely within 30 s of vortexing |
| Example 2 | 1 h | 330.96 | 0.172 | 2 | 227.64 | 339.52 | 646.64 | |
| | 2 h | 356.33 | 0.201 | 2 | 194.46 | 361.19 | 950.46 | |
| Comparative | 0 h | 354.07 | 0.252 | 2 | 246.82 | 379.59 | 507.28 | Granules dissolved completely within 30 s of vortexing |
| Example 3 | 1 h | 379.28 | 0.268 | 2 | 266.36 | 391.26 | 863.41 | |
| | 2 h | 450.65 | 0.293 | 3 | 221.75 | 439.51 | 2019.7 | |
| Comparative | 0 h | 312.57 | 0.137 | 2 | 217.35 | 325.48 | 446.81 | Granules dissolved completely within 30 s of vortexing |
| Example 4 | 1 h | 327.91 | 0.166 | 2 | 219.67 | 337.22 | 637.12 | |
| | 2 h | 353.28 | 0.175 | 2 | 200.23 | 396.25 | 890.56 | |
| Comparative | 0 h | 292.94 | 0.127 | 2 | 193.04 | 299.10 | 436.44 | Granules dissolved completely within 30 s of vortexing |
| Example 5 | 1 h | 301.03 | 0.119 | 2 | 206.96 | 305.84 | 442.21 | |
| | 2 h | 319.40 | 0.202 | 2 | 173.78 | 319.76 | 502.17 | |
| Comparative | 0 h | 316.23 | 0.141 | 2 | 216.79 | 329.10 | 456.09 | Granules dissolved completely within 30 s of vortexing |
| Example 6 | 1 h | 325.68 | 0.169 | 2 | 224.91 | 343.99 | 643.13 | |
| | 2 h | 363.06 | 0.188 | 2 | 198.62 | 379.97 | 921.76 | |
| Comparative | 0 h | 178.06 | 0.118 | 2 | 80.79 | 171.67 | 305.82 | Granules dissolved completely within 30 s of vortexing |
| Example 7 | 1 h | 174.82 | 0.117 | 2 | 80.39 | 175.87 | 307.12 | |
| | 2 h | 190.02 | 0.140 | 2 | 59.82 | 190.58 | 369.97 | |
| Comparative | 0 h | 176.32 | 0.129 | 2 | 81.54 | 172.14 | 320.24 | Granules dissolved completely within 30 s of vortexing |
| Example 8 | 1 h | 176.12 | 0.111 | 2 | 89.02 | 173.75 | 295.96 | |
| | 2 h | 191.96 | 0.137 | 2 | 60.57 | 177.47 | 384.06 | |
| Comparative | 0 h | 180.25 | 0.120 | 2 | 82.87 | 176.70 | 313.26 | Granules dissolved completely within 30 s of vortexing |
| Example 9 | 1 h | 179.62 | 0.115 | 2 | 84.6 | 179.75 | 308.10 | |
| | 2 h | 192.57 | 0.149 | 2 | 61.57 | 185.21 | 389.92 | |
| Comparative | 0 h | 308.67 | 0.434 | 3 | 200.15 | 358.36 | 617.37 | Granules dissolved completely within 30 s of vortexing |
| Example 10 | 1 h | 1019.96 | 0.431 | 3 | 548.37 | 895.26 | 9576.52 | |
| | 2 h | 1593.69 | 0.537 | 3 | 669.56 | 1409.60 | 11063.04 | |

### 3. Storage Stability Test Method:

### Related Substances

Solvent: Acetonitrile-water (50:50, v/v)

Test Solution: An appropriate amount of the test sample was taken, weighed accurately, dissolved in the solvent and diluted to a solution containing about 0.1 mg of the test sample per ml.

Reference Standard Solution: Appropriate amounts of isavuconazole, Impurity B, Impurity D, Impurity E, Impurity F, Impurity G, Impurity H, Impurity I, Impurity J, and Impurity K were taken as the reference standards, weighed accurately, dissolved in the solvent and diluted to a solution containing about 2 μg of each substance per ml.

Sensitivity Solution: An appropriate amount of isavuconazole reference standard was taken, dissolved in the solvent and diluted to a solution containing about 0.1 μg of isavuconazole reference standard per ml.

Chromatographic Conditions: Octadecylsilane-bonded silica gel as the packing material of column (such as Ultimate XB-C18, 4.6 mm×250 mm, 5 μm or a column with an equivalent performance); a trapping column (such as Welch Ghost-Buster, 4.6 mm×50 mm; or a trapping column with an equivalent performance) connected between the mixer and the injector; 0.05% phosphoric acid solution (0.5 ml of phosphoric acid was taken and diluted to 1000 ml with water) as the mobile phase A, and acetonitrile-water (80:20 v/v) as the mobile phase B. Flow rate: 1.0 ml/min; gradient elution as shown in Table 10; Detection wavelength: 210 nm; Column temperature: 35° C.; and Injection volume: 10 μl.

TABLE 10

| Time (minutes) | Mobile Phase A (%) | Mobile Phase B (%) |
|---|---|---|
| 0 | 80 | 20 |
| 10 | 80 | 20 |
| 20 | 70 | 30 |
| 25 | 70 | 30 |
| 50 | 10 | 90 |
| 65 | 10 | 90 |
| 66 | 80 | 20 |
| 80 | 80 | 20 |

System Suitability Requirements: In the chromatogram of the reference standard solution, the peaks are in the order of impurity G, impurity H, impurity I, impurity F, impurity D, impurity K, impurity B, isavuconazole, and impurity J. The resolution between adjacent peaks shall meet the specified requirements. In the chromatogram of the sensitivity solution, the signal-to-noise ratio of the principal component peak shall not be less than 10.

Procedure: The test solution and the reference standard solution were injected into the liquid chromatograph, and the chromatograms were recorded.

Limits: In the chromatogram of the test solution, excluding excipient peaks and solvent peaks, with Area Normalization Method, the area of impurity B, impurity D, impurity E, impurity F, impurity G, impurity H, impurity I, impurity J, impurity K, and the impurity eluted at about 0.97 relative retention time (RRT) of the main peak is not greater than 0.2%. The area of any other individual peak is not greater than 0.2%. The sum of the areas of all impurities is not greater than 2.0%. Disregard any peak with an area less than the area of the principal peak in the chromatogram obtained with the sensitivity solution.

Granules were sealed in packaging and placed in an oven at 50° C. for 1 month. Samples were taken at 0 day and 1 month, and related substances were determined. Impurity contents were calculated using the area normalization method, with impurities less than 0.01% disregarded.

The storage stability test results of the pharmaceutical compositions in granule form as prepared in Examples 1-23 and Comparative Examples 1-10 were shown in Table 11.

TABLE 11

| | | Maximum | | | | | | | | | | |
|---|---|---|---|---|---|---|---|---|---|---|---|---|
| | | Unknown | | | | | | | | | | |
| | | Individual | | | | | | | | | | Total |
| | Sampling | Impurity | | | | | | | | | | Impurities |
| Sample | Time | (%)% | ImpG % | ImpH % | ImpI % | ImpF % | ImpD % | ImpK % | ImpB % | RRT0.97% | ImpJ % | % |
| Example 1 | Day 0 | 0.020 | 0.025 | Not | Not | Not | Not | 0.023 | Not | 0.022 | 0.011 | 0.146 |
| | | | | detected | detected | detected | detected | | detected | | | |
| | 1 Month | 0.021 | 0.028 | Not | Not | Not | Not | 0.027 | Not | 0.025 | 0.011 | 0.154 |
| | | | | detected | detected | detected | detected | | detected | | | |
| Example 2 | Day 0 | 0.020 | 0.022 | Not | Not | Not | Not | 0.023 | Not | 0.019 | 0.011 | 0.144 |
| | | | | detected | detected | detected | detected | | detected | | | |
| | 1 Month | 0.022 | 0.019 | Not | Not | Not | Not | 0.026 | Not | 0.023 | 0.012 | 0.149 |
| | | | | detected | detected | detected | detected | | detected | | | |
| Example 3 | Day 0 | 0.022 | 0.022 | Not | Not | Not | Not | 0.024 | Not | 0.021 | 0.011 | 0.142 |
| | | | | detected | detected | detected | detected | | detected | | | |
| | 1 Month | 0.023 | 0.022 | Not | Not | Not | Not | 0.025 | Not | 0.023 | 0.012 | 0.148 |
| | | | | detected | detected | detected | detected | | detected | | | |
| Example 4 | Day 0 | 0.018 | 0.030 | Not | Not | Not | Not | 0.023 | Not | 0.022 | 0.011 | 0.146 |
| | | | | detected | detected | detected | detected | | detected | | | |
| | 1 Month | 0.021 | 0.028 | Not | Not | Not | Not | 0.027 | Not | 0.025 | 0.011 | 0.154 |
| | | | | detected | detected | detected | detected | | detected | | | |
| Example 5 | Day 0 | 0.021 | 0.024 | Not | Not | Not | Not | 0.018 | Not | 0.017 | 0.013 | 0.152 |
| | | | | detected | detected | detected | detected | | detected | | | |
| | 1 Month | 0.020 | 0.027 | Not | Not | Not | Not | 0.024 | Not | 0.023 | 0.011 | 0.163 |
| | | | | detected | detected | detected | detected | | detected | | | |
| Example 6 | Day 0 | 0.016 | 0.027 | Not | Not | Not | Not | 0.024 | Not | 0.020 | 0.011 | 0.149 |
| | | | | detected | detected | detected | detected | | detected | | | |
| | 1 Month | 0.019 | 0.025 | Not | Not | Not | Not | 0.027 | Not | 0.025 | 0.011 | 0.153 |
| | | | | detected | detected | detected | detected | | detected | | | |
| Example 7 | Day 0 | 0.019 | 0.027 | Not | Not | Not | Not | 0.022 | Not | 0.021 | 0.012 | 0.150 |
| | | | | detected | detected | detected | detected | | detected | | | |
| | 1 Month | 0.024 | 0.028 | Not | Not | Not | Not | 0.024 | Not | 0.026 | 0.011 | 0.161 |
| | | | | detected | detected | detected | detected | | detected | | | |
| Comparative | Day 0 | 0.022 | 0.023 | Not | Not | Not | Not | 0.024 | Not | 0.024 | 0.012 | 0.152 |
| Example 1 | | | | detected | detected | detected | detected | | detected | | | |
| | 1 Month | 0.024 | 0.019 | Not | Not | Not | Not | 0.025 | Not | 0.026 | 0.011 | 0.155 |
| | | | | detected | detected | detected | detected | | detected | | | |
| Comparative | Day 0 | 0.019 | 0.024 | Not | Not | Not | Not | 0.019 | Not | 0.022 | 0.012 | 0.149 |
| Example 2 | | | | detected | detected | detected | detected | | detected | | | |
| | 1 Month | 0.022 | 0.023 | Not | Not | Not | Not | 0.024 | Not | 0.023 | 0.012 | 0.153 |
| | | | | detected | detected | detected | detected | | detected | | | |
| Comparative | Day 0 | 0.023 | 0.022 | Not | Not | Not | Not | 0.024 | Not | 0.026 | 0.013 | 0.149 |
| Example 3 | | | | detected | detected | detected | detected | | detected | | | |
| | 1 Month | 0.022 | 0.000 | Not | Not | Not | Not | 0.025 | Not | 0.024 | 0.011 | 0.155 |
| | | | | detected | detected | detected | detected | | detected | | | |
| Comparative | Day 0 | 0.019 | 0.024 | Not | Not | Not | Not | 0.020 | Not | 0.024 | 0.011 | 0.147 |
| Example 4 | | | | detected | detected | detected | detected | | detected | | | |
| | 1 Month | 0.023 | 0.026 | Not | Not | Not | Not | 0.024 | Not | 0.022 | 0.013 | 0.016 |
| | | | | detected | detected | detected | detected | | detected | | | |
| Comparative | Day 0 | 0.019 | 0.026 | Not | Not | Not | Not | 0.023 | Not | 0.021 | 0.011 | 0.152 |
| Example 5 | | | | detected | detected | detected | detected | | detected | | | |
| | 1 Month | 0.023 | 0.029 | Not | Not | Not | Not | 0.022 | Not | 0.024 | 0.012 | 0.160 |
| | | | | detected | detected | detected | detected | | detected | | | |
| Comparative | Day 0 | 0.021 | 0.025 | Not | Not | Not | Not | 0.021 | Not | 0.022 | 0.011 | 0.148 |
| Example 6 | | | | detected | detected | detected | detected | | detected | | | |
| | 1 Month | 0.023 | 0.027 | Not | Not | Not | Not | 0.023 | Not | 0.024 | 0.012 | 0.156 |
| | | | | detected | detected | detected | detected | | detected | | | |
| Comparative | Day 0 | 0.017 | 0.031 | Not | Not | Not | Not | 0.031 | Not | 0.016 | 0.012 | 0.142 |
| Example 7 | | | | detected | detected | detected | detected | | detected | | | |
| | 1 Month | 0.357 | 0.524 | 0.012 | 0.011 | Not | Not | 0.105 | Not | 0.026 | 0.011 | 1.462 |
| | | | | | | detected | detected | | detected | | | |
| Example 8 | Day 0 | 0.020 | 0.022 | Not | Not | Not | Not | 0.019 | Not | 0.018 | 0.010 | 0.136 |
| | | | | detected | detected | detected | detected | | detected | | | |
| | 1 Month | 0.022 | 0.018 | Not | Not | Not | Not | 0.024 | Not | 0.022 | 0.012 | 0.143 |
| | | | | detected | detected | detected | detected | | detected | | | |
| Example 9 | Day 0 | 0.023 | 0.026 | Not | Not | Not | Not | 0.022 | Not | 0.023 | 0.014 | 0.149 |
| | | | | detected | detected | detected | detected | | detected | | | |
| | 1 Month | 0.023 | 0.025 | Not | Not | Not | Not | 0.025 | Not | 0.026 | 0.011 | 0.152 |
| | | | | detected | detected | detected | detected | | detected | | | |
| Example 10 | Day 0 | 0.019 | 0.024 | Not | Not | Not | Not | 0.022 | Not | 0.021 | 0.011 | 0.142 |
| | | | | detected | detected | detected | detected | | detected | | | |
| | 1 Month | 0.021 | 0.022 | Not | Not | Not | Not | 0.026 | Not | 0.024 | 0.013 | 0.147 |
| | | | | detected | detected | detected | detected | | detected | | | |
| Comparative | Day 0 | 0.018 | 0.030 | Not | Not | Not | Not | 0.024 | Not | 0.019 | 0.012 | 0.150 |
| Example 8 | | | | detected | detected | detected | detected | | detected | | | |
| | 1 Month | 0.152 | 0.031 | 0.083 | 0.017 | Not | Not | 0.028 | Not | 0.024 | 0.013 | 0.556 |
| | | | | | | detected | detected | | detected | | | |
| Comparative | Day 0 | 0.016 | 0.019 | Not | Not | Not | Not | 0.019 | Not | 0.022 | 0.011 | 0.139 |
| Example 9 | | | | detected | detected | detected | detected | | detected | | | |
| | 1 Month | 0.161 | 0.018 | 0.056 | 0.042 | Not | Not | 0.022 | Not | 0.026 | 0.012 | 0.531 |
| | | | | | | detected | detected | | detected | | | |
| Example 11 | Day 0 | 0.020 | 0.032 | Not | Not | Not | Not | 0.020 | Not | 0.017 | 0.013 | 0.150 |
| | | | | detected | detected | detected | detected | | detected | | | |
| | 1 Month | 0.021 | 0.028 | Not | Not | Not | Not | 0.024 | Not | 0.018 | 0.010 | 0.153 |
| | | | | detected | detected | detected | detected | | detected | | | |
| Example 12 | Day 0 | 0.022 | 0.023 | Not | Not | Not | Not | 0.023 | Not | 0.022 | 0.012 | 0.152 |
| | | | | detected | detected | detected | detected | | detected | | | |
| | 1 Month | 0.025 | 0.026 | Not | Not | Not | Not | 0.022 | Not | 0.024 | 0.012 | 0.154 |
| | | | | detected | detected | detected | detected | | detected | | | |
| Example 13 | Day 0 | 0.022 | 0.021 | Not | Not | Not | Not | 0.025 | Not | 0.022 | 0.011 | 0.148 |
| | | | | detected | detected | detected | detected | | detected | | | |
| | 1 Month | 0.021 | 0.023 | Not | Not | Not | Not | 0.024 | Not | 0.023 | 0.011 | 0.154 |
| | | | | detected | detected | detected | detected | | detected | | | |
| Comparative | Day 0 | 0.019 | 0.023 | Not | Not | Not | Not | 0.021 | Not | 0.025 | 0.012 | 0.147 |
| Example 14 | | | | detected | detected | detected | detected | | detected | | | |
| | 1 Month | 0.022 | 0.023 | Not | Not | Not | Not | 0.024 | Not | 0.021 | 0.012 | 0.150 |
| | | | | detected | detected | detected | detected | | detected | | | |
| Example 15 | Day 0 | 0.023 | 0.022 | Not | Not | Not | Not | 0.025 | Not | 0.023 | 0.012 | 0.153 |
| | | | | detected | detected | detected | detected | | detected | | | |
| | 1 Month | 0.025 | 0.026 | Not | Not | Not | Not | 0.027 | Not | 0.025 | 0.013 | 0.160 |
| | | | | detected | detected | detected | detected | | detected | | | |
| Example 16 | Day 0 | 0.022 | 0.026 | Not | Not | Not | Not | 0.023 | Not | 0.022 | 0.012 | 0.155 |
| | | | | detected | detected | detected | detected | | detected | | | |
| | 1 Month | 0.025 | 0.023 | Not | Not | Not | Not | 0.026 | Not | 0.022 | 0.011 | 0.160 |
| | | | | detected | detected | detected | detected | | detected | | | |
| Example 17 | Day 0 | 0.022 | 0.024 | Not | Not | Not | Not | 0.021 | Not | 0.025 | 0.011 | 0.148 |
| | | | | detected | detected | detected | detected | | detected | | | |
| | 1 Month | 0.024 | 0.027 | Not | Not | Not | Not | 0.024 | Not | 0.027 | 0.012 | 0.157 |
| | | | | detected | detected | detected | detected | | detected | | | |
| Example 18 | Day 0 | 0.019 | 0.021 | Not | Not | Not | Not | 0.022 | Not | 0.021 | 0.011 | 0.149 |
| | | | | detected | detected | detected | detected | | detected | | | |
| | 1 Month | 0.021 | 0.023 | Not | Not | Not | Not | 0.025 | Not | 0.024 | 0.012 | 0.155 |
| | | | | detected | detected | detected | detected | | detected | | | |
| Example 19 | Day 0 | 0.022 | 0.019 | Not | Not | Not | Not | 0.021 | Not | 0.022 | 0.012 | 0.152 |
| | | | | detected | detected | detected | detected | | detected | | | |
| | 1 Month | 0.022 | 0.022 | Not | Not | Not | Not | 0.024 | Not | 0.022 | 0.011 | 0.159 |
| | | | | detected | detected | detected | detected | | detected | | | |
| Example 20 | Day 0 | 0.023 | 0.025 | Not | Not | Not | Not | 0.021 | Not | 0.021 | 0.011 | 0.149 |
| | | | | detected | detected | detected | detected | | detected | | | |
| | 1 Month | 0.026 | 0.028 | Not | Not | Not | Not | 0.023 | Not | 0.022 | 0.013 | 0.153 |
| | | | | detected | detected | detected | detected | | detected | | | |
| Example 21 | Day 0 | 0.022 | 0.025 | Not | Not | Not | Not | 0.019 | Not | 0.025 | 0.013 | 0.146 |
| | | | | detected | detected | detected | detected | | detected | | | |
| | 1 Month | 0.024 | 0.023 | Not | Not | Not | Not | 0.021 | Not | 0.024 | 0.011 | 0.151 |
| | | | | detected | detected | detected | detected | | detected | | | |
| Example 22 | Day 0 | 0.019 | 0.024 | Not | Not | Not | Not | 0.022 | Not | 0.023 | 0.011 | 0.149 |
| | | | | detected | detected | detected | detected | | detected | | | |
| | 1 Month | 0.021 | 0.026 | Not | Not | Not | Not | 0.022 | Not | 0.022 | 0.011 | 0.156 |
| | | | | detected | detected | detected | detected | | detected | | | |
| Example 23 | Day 0 | 0.023 | 0.024 | Not | Not | Not | Not | 0.019 | Not | 0.022 | 0.012 | 0.152 |
| | | | | detected | detected | detected | detected | | detected | | | |
| | 1 Month | 0.021 | 0.022 | Not | Not | Not | Not | 0.021 | Not | 0.025 | 0.013 | 0.157 |
| | | | | detected | detected | detected | detected | | detected | | | |
| Comparative | Day 0 | 0.023 | 0.026 | Not | Not | Not | Not | 0.022 | Not | 0.021 | 0.011 | 0.152 |
| Example 10 | | | | detected | detected | detected | detected | | detected | | | |
| | 1 Month | 0.022 | 0.024 | Not | Not | Not | Not | 0.022 | Not | 0.023 | 0.013 | 0.156 |
| | | | | detected | detected | detected | detected | | detected | | | |

The structures of impurities B, D, F, G, H, I, J and K in Table 11 were shown in Table 12.

TABLE 12

| Impurity Name | Structure |
|---|---|
| Impurity B | 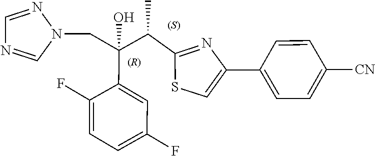 Chemical Formula: C₂₂H₁₇F₂N₅OS Molecular Weight: 437.47 |
| Impurity D | 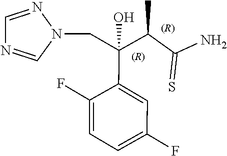 Chemical Formula: C₁₃H₁₄F₂N₄OS Molecular Weight: 312.34 |
| Impurity F | 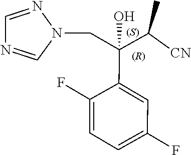 Chemical Formula: C₁₃H₁₂F₂N₄O Molecular Weight: 278.26 |
| Impurity G | 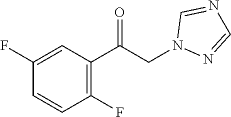 [SMILES: O=C(Cn1cncn1)c1cc(F)ccc1F] Chemical Formula: C₁₀H₇F₂N₃O Molecular Weight: 223.18 |
| Impurity H | 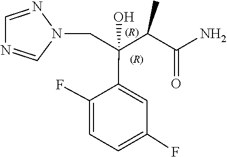 Chemical Formula: C₁₃H₁₄F₂N₄O₂ Molecular Weight: 296.28 |
| Impurity I | 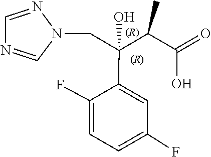 Chemical Formula: C₁₃H₁₃F₂N₃O₃ Molecular Weight: 297.26 |
| Impurity J | 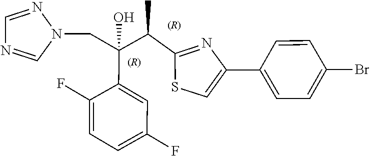 Chemical Formula: C₂₁H₁₇BrF₂N₄OS Molecular Weight: 491.35 |
| Impurity K | 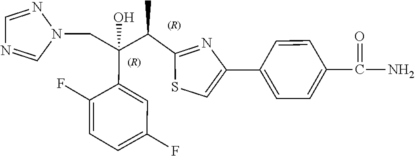 Chemical Formula: C₂₂H₁₉F₂N₅O₂S Molecular Weight: 455.48 |

2.5 g of each of the pharmaceutical compositions in granule form as prepared in Example 1 and Comparative Examples 1 to 6 were placed into the sealed polyester/aluminum/polyethylene composite film pouches, respectively. After storage at 40° C. and 75% relative humidity, their characteristics at 15 day and 1 month were shown in Table 13.

TABLE 13

| Sample | Day 0 | 40° C., Day 15 | 40° C., 1 month |
|---|---|---|---|
| Example 1 | Off-white | Off-white granules | Off-white |
| | granules | | granules |
| Comparative | Off-white | Off-white granules | Off-white |
| Example 1 | granules | | granules |
| Comparative | Off-white | Off-white granules | Off-white |
| Example 2 | granules | | granules |
| Comparative | Off-white | Off-white granules | Off-white |
| Example 3 | granules | | granules |
| Comparative | Off-white | Off-white granules | Off-white |
| Example 4 | granules | | granules |
| Comparative | Off-white | Off-white granules with a | Off-white |
| Example 5 | granules | small amount of cakes | cakes |
| Comparative | Off-white | Off-white granules with a | Off-white |
| Example 6 | granules | small amount of cakes | cakes |

Conclusion: As shown in Tables 8 to 13, the pharmaceutical compositions of Examples 1 to 23 exhibited excellent dissolution, particle size stability and storage stability. In contrast, the pharmaceutical compositions of Comparative Examples 1 to 4 and 6 exhibited good particle size stability and storage stability but low dissolution; the pharmaceutical composition of Comparative Example 5 exhibited good dissolution but poor storage stability; the pharmaceutical compositions of Comparative Examples 7 to 9 exhibited good dissolution and particle size stability but poor storage stability; and the pharmaceutical composition of Comparative Example 10 exhibited good dissolution and storage stability but poor particle size stability. It was demonstrated that the pharmaceutical compositions of the present disclosure had excellent dissolution, particle size stability, and storage stability.

### 4. Pharmacokinetic Test Method

Study on Tissue Distribution and Plasma Pharmacokinetics in SD Rats after Oral Administration of Isavuconazole, Isavuconazonium Sulfate, and the Pharmaceutical Compositions of Examples 1 and 2.

Method: Sixty SD rats (male) were randomly divided into 4 groups, with 15 rats in each group, and were administered by gavage with isavuconazole API (Group 1), isavuconazonium sulfate capsule (Group 2, Manufacturer: SwissCo Service AG, MAH: Pfizer Australia Pty Ltd, Lot No.: W077382A), the pharmaceutical composition of Example 1 (Group 3) and the pharmaceutical composition of Example 2 (Group 4), respectively. The animals were euthanized at 1 h, 2 h, 4 h, 8 h, and 24 h after administration, and kidney, bladder, and urine samples were collected (3 animals per group at each time point). Blood samples were also collected at 0.25 h, 0.5 h, 1 h, 2 h, 4 h, 6 h, 8 h, 12 h, and 24 h (from animals at the 24 h time point). Samples were analyzed to determine drug concentrations, calculate pharmacokinetic parameters and compare intergroup differences.

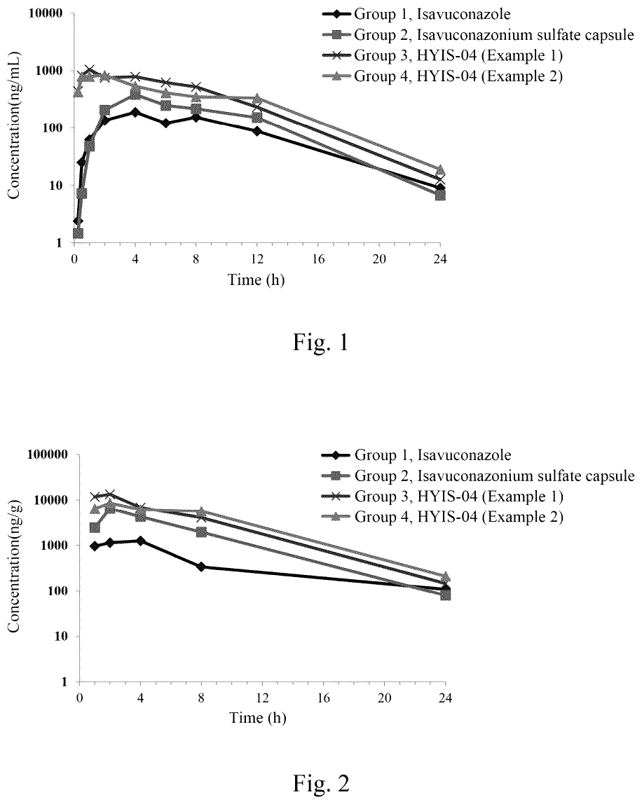

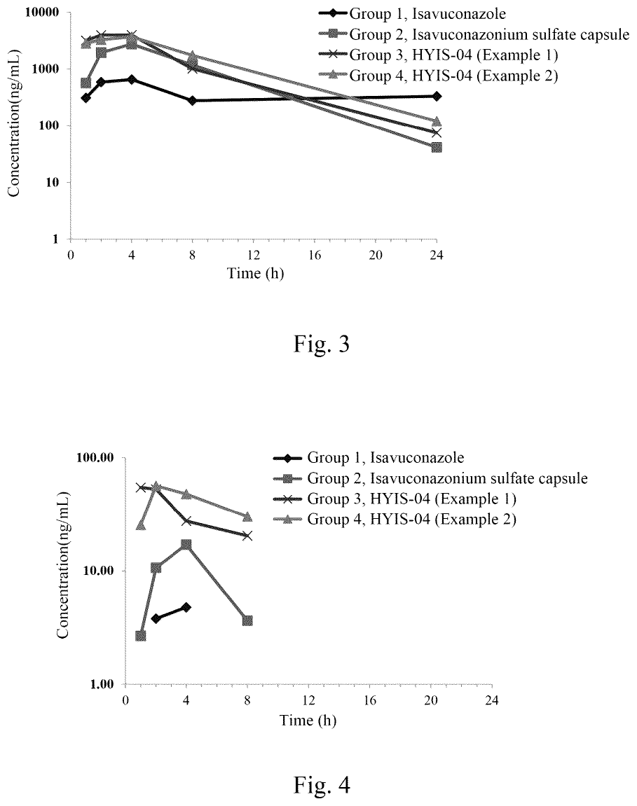

Results: Referring to FIG. 1, AUC values of isavuconazole in the plasma for Groups 1-4 were 2090 ng/ml h, 3090 ng/ml h, 8600 ng/ml h, and 7930 ng/mL h, respectively. Referring to FIG. 2, AUC values of isavuconazole in the kidney for Groups 1-4 were 10100 ng/g·h, 43500 ng/g·h, 87500 ng/g·h, and 92200 ng/g·h, respectively. Referring to FIG. 3, AUC values of isavuconazole in the bladder for Groups 1-4 were 8360 ng/g·h, 23400 ng/g·h, 29900 ng/g·h, and 35600 ng/g·h, respectively. Referring to FIG. 4, AUC values of isavuconazole in the urine for Groups 1-4 were 8.51 ng/mL·h, 75.4 ng/mL·h, 229 ng/mL·h, and 300 ng/mL·h, respectively.

Plasma and tissue distribution: Isavuconazole exhibited the highest concentration in the kidney, followed by the bladder and plasma. Due to inaccurate collection of urine samples, this exposure was the lowest.

Conclusion: Following oral administration of the equivalent dose of 20 mg/kg (calculated as isavuconazole) to SD rats, the pharmaceutical compositions of Examples 1 and 2 exhibited higher concentrations than both the isavuconazonium sulfate capsule and the isavuconazole API in plasma, kidney, bladder, and urine.

Finally, it should be noted that the above examples are provided solely to illustrate the technical solutions of the present disclosure, and are not intended to limit the scope of the present disclosure. Although the present disclosure has been described in detail with reference to the foregoing examples, it will be understood by a person skilled in the art that modifications can be made to the technical solutions described in the foregoing examples, or equivalent replacements can be made to some or all of the technical features therein; such modifications or replacements do not cause the corresponding technical solutions to depart from the scope of the embodiments and examples of the present disclosure, and they should all be encompassed within the scope of the claims and the description of the present disclosure. In particular, provided there is no technical conflict, various technical features mentioned in the above embodiments can be combined in any manner. The present disclosure is not limited to the specific examples disclosed herein, but encompasses all technical solutions falling within the scope of the claims.

What is claimed is:

1. A pharmaceutical composition, comprising isavuconazole, a first nonionic surfactant, a diluent, a stabilizer, and a degradation inhibitor, wherein the first nonionic surfactant comprises copovidone and poloxamer.

2. The pharmaceutical composition according to claim 1, wherein a mass ratio of the isavuconazole, the copovidone, the poloxamer, the diluent, the stabilizer, and the degradation inhibitor is 1:2.5-4.5:0.45-1:3-5:3.5-5.5:0.25-2.

3. The pharmaceutical composition according to claim 1, further comprising a second nonionic surfactant, wherein second nonionic surfactant comprises more of Polyoxyl(40)Hydrogenated Castor Oil (RH40), TPGS, and HS15; and
optionally, a mass ratio of the isavuconazole to the second nonionic surfactant is 1:0.1-2.

4. The pharmaceutical composition according to claim 2, further comprising a second nonionic surfactant, wherein the second nonionic surfactant comprises one or more of Polyoxyl(40)Hydrogenated Castor Oil (RH40), TPGS, and HS15; and
optionally, a mass ratio of the isavuconazole to the second nonionic surfactant is 1:0.1-2.

5. The pharmaceutical composition according to claim 1, further comprising an ionic surfactant;
optionally, a mass ratio of the isavuconazole to the ionic surfactant is 1:0.5-1;
optionally, the stabilizer comprises one or more of Hypromellose E5, Hypromellose E50, Hypromellose K100LV, Hypromellose E4M, Hypromellose K4M, hydroxypropyl cellulose LF, hydroxypropyl cellulose LXF, hydroxypropyl cellulose JF, hydroxypropyl cellulose JXF, hydroxypropyl cellulose HXF, hydroxypropyl cellulose GXF, and hydroxypropyl cellulose GF; alternatively, the stabilizer comprises one or more of Hypromellose E5 and Hypromellose E50; and
optionally, the ionic surfactant comprises one or more of sodium glycocholate, lecithin, and sodium dodecyl sulfate.

6. The pharmaceutical composition according to claim 2, further comprising an ionic surfactant;
optionally, a mass ratio of the isavuconazole to the ionic surfactant is 1:0.5-1;
optionally, the stabilizer comprises one or more of Hypromellose E5, Hypromellose E50, Hypromellose K100LV, Hypromellose E4M, Hypromellose K4M, hydroxypropyl cellulose LF, hydroxypropyl cellulose LXF, hydroxypropyl cellulose JF, hydroxypropyl cellulose JXF, hydroxypropyl cellulose HXF, hydroxypropyl cellulose GXF, and hydroxypropyl cellulose GF; alternatively, the stabilizer comprises one or more of Hypromellose E5 and Hypromellose E50; and
optionally, the ionic surfactant comprises one or more of sodium glycocholate, lecithin, and sodium dodecyl sulfate.

7. The pharmaceutical composition according to claim 4, further comprising an ionic surfactant;
optionally, a mass ratio of the isavuconazole to the ionic surfactant is 1:0.5-1;
optionally, the stabilizer comprises one or more of Hypromellose E5, Hypromellose E50, Hypromellose K100LV, Hypromellose E4M, Hypromellose K4M, hydroxypropyl cellulose LF, hydroxypropyl cellulose LXF, hydroxypropyl cellulose JF, hydroxypropyl cellulose JXF, hydroxypropyl cellulose HXF, hydroxypropyl cellulose GXF, and hydroxypropyl cellulose GF; alternatively, the stabilizer comprises one or more of Hypromellose E5 and Hypromellose E50; and
optionally, the ionic surfactant comprises one or more of sodium glycocholate, lecithin, and sodium dodecyl sulfate.

8. The pharmaceutical composition according to claim 1, further comprising one or more of a flavoring agent, a flavor and an oily solvent.

9. The pharmaceutical composition according to claim 2, further comprising one or more of a flavoring agent, a flavor and an oily solvent.

10. The pharmaceutical composition according to claim 4, further comprising one or more of a flavoring agent, a flavor and an oily solvent.

11. The pharmaceutical composition according to claim 6, further comprising one or more of a flavoring agent, a flavor and an oily solvent.

12. The pharmaceutical composition according to claim 8, wherein a mass ratio of the isavuconazole to the flavoring agent is 1:0.1-2;
optionally, the flavor is present at a content of 0.01% to 2%, based on the total mass of the pharmaceutical composition; and
optionally, the oily solvent is present at a content of 2% to 6%, based on the total mass of the pharmaceutical composition.

13. The pharmaceutical composition according to claim 9, wherein a mass ratio of the isavuconazole to the flavoring agent is 1:0.1-2;
optionally, the flavor is present at a content of 0.01% to 2%, based on the total mass of the pharmaceutical composition; and
optionally, the oily solvent is present at a content of 2% to 6%, based on the total mass of the pharmaceutical composition.

14. The pharmaceutical composition according to claim 1, wherein the diluent comprises one or more of crospovidone, microcrystalline cellulose, silicon dioxide, pregelatinized starch, corn starch, lactose, xylitol, and mannitol; and
optionally, the degradation inhibitor comprises one or more of anhydrous citric acid, anhydrous citric acid-sodium hydroxide, monosodium citrate, tartaric acid-sodium hydroxide, sodium bitartrate, succinic acid-sodium hydroxide, sodium bisuccinate, malic acid-sodium hydroxide, monosodium malate, benzoic acid, and citric acid-trisodium citrate.

15. The pharmaceutical composition according to claim 2, wherein the diluent comprises one or more of crospovidone, microcrystalline cellulose, silicon dioxide, pregelatinized starch, corn starch, lactose, xylitol, and mannitol; and
optionally, the degradation inhibitor comprises one or more of anhydrous citric acid, anhydrous citric acid-sodium hydroxide, monosodium citrate, tartaric acid-sodium hydroxide, sodium bitartrate, succinic acid-sodium hydroxide, sodium bisuccinate, malic acid-sodium hydroxide, monosodium malate, benzoic acid, and citric acid-trisodium citrate.

16. The pharmaceutical composition according to claim 4, wherein the diluent comprises one or more of crospovidone, microcrystalline cellulose, silicon dioxide, pregelatinized starch, corn starch, lactose, xylitol, and mannitol; and
optionally, the degradation inhibitor comprises one or more of anhydrous citric acid, anhydrous citric acid-sodium hydroxide, monosodium citrate, tartaric acid-sodium hydroxide, sodium bitartrate, succinic acid-sodium hydroxide, sodium bisuccinate, malic acid-sodium hydroxide, monosodium malate, benzoic acid, and citric acid-trisodium citrate.

17. The pharmaceutical composition according to claim 6, wherein the diluent comprises one or more of crospovidone, microcrystalline cellulose, silicon dioxide, pregelatinized starch, corn starch, lactose, xylitol, and mannitol; and
optionally, the degradation inhibitor comprises one or more of anhydrous citric acid, anhydrous citric acid-sodium hydroxide, monosodium citrate, tartaric acid-sodium hydroxide, sodium bitartrate, succinic acid-sodium hydroxide, sodium bisuccinate, malic acid-sodium hydroxide, monosodium malate, benzoic acid, and citric acid-trisodium citrate.

18. The pharmaceutical composition according to claim 1, wherein a dosage form of the pharmaceutical composition includes granules, a tablet, a powder, an emulsion, a solution or a suspension.

19. A method for preparing the pharmaceutical composition according to claim 1, comprising mixing isavuconazole, a first nonionic surfactant, a diluent, a stabilizer, a degradation inhibitor, and a solvent, wherein the first nonionic surfactant comprises copovidone and poloxamer.

20. A method for treating or ameliorating a disease selected from fungal urinary tract infection, candidal vaginitis, aspergillosis and mucormycosis in a subject in need thereof, comprising administering the pharmaceutical composition according to claim 1 to the subject.
